(19) United States
(12) Patent Application Publication
(10) Pub. No.: US 2026/0083768 A1
(43) Pub. Date: Mar. 26, 2026

(54) INFLAMMASOME INHIBITION FOR NEUROPROTECTION

(71) Applicants: Jayakrishna AMBATI, Charlottesville, VA (US); Peirong HUANG, Shanghai (CN); Kameshwari AMBATI, Charlottesville, VA (US); University of Virginia Patent Foundation, Charlottesville, VA (US); UNIVESITY OF VIRGINIA, Charlottesville, VA (US)

(72) Inventors: Jayakrishna Ambati, Charlottesville, VA (US); Peirong Huang, Shanghai (CN); Kameshwari Ambati, Charlottesville, VA (US)

(21) Appl. No.: 19/109,969

(22) PCT Filed: Sep. 8, 2023
(86) PCT No.: PCT/US2023/073779

§ 371 Date: Mar. 7, 2025

Related U.S. Application Data
(60) Provisional application No. 63/375,144, filed on Sep. 9, 2022.
(60) Provisional application No. 63/479,586, filed on Jan. 12, 2023.

(51) Int. Cl. A61K 31/7072; A61K 31/513; A61P 27/02

(52) U.S. Cl. CPC A61K 31/7072; A61K 31/513; A61P 27/02

(57) ABSTRACT

Provided herein are methods to treat rhegmatogenous retinal detachment (RRD) and/or improve visual outcomes of RRD.

### PRIORITY

This application claims priority to and the benefit of U.S. Provisional Patent Application Nos. 63/375,144, filed Sep. 9, 2022; and 63/479,586, filed Jan. 12, 2023, the entire disclosures of which are incorporated herein by this reference.

### BACKGROUND

Rhegmatogenous retinal detachment (RRD) is a serious and relatively common ocular pathology. Incidence estimates of RRD range from 6.3 to 17.9 per 100,000 persons (1). RRD results from escape of vitreous humor through a retinal tear into the subretinal space, separating the neurosensory retina from the underlying retinal pigmented epithelium (RPE). Once detached, photoreceptors are vulnerable to hypoxia and damaging inflammatory signaling, which hasten photoreceptor apoptosis and vision loss. (2-6) In this emergent context, prompt therapeutic action is needed to minimize lasting damage.

Currently, RRD management hinges on surgical repair. Even with successful reattachment, however, many patients suffer lasting visual deficits. (7) In “macula-off” RRD repaired within nine days, only half of patients may regain 20/50 vision. (8) Visual outcomes worsen with increasing delay in surgical repair. For example, only one-third of patients who undergo surgery after 10-19 days regain 20/50 vision, (8) and even delays of more than 3 days have been reported to result in worse visual outcomes. (9) While detached, damage to the retina leaves residual functional deficits even after anatomic repair. Therefore, a targeted therapy is needed to provide protective benefits while patients await RRD surgical repair.

### SUMMARY

Retinal detachment is a cause of vision loss and blindness. Surgical repair is the current standard of care for this disorder; however, delays in surgery can lead to persistent vision loss despite successful surgical reattachment of the retina. This is due to death of photoreceptors, the cells in the retina that are responsible for sensing light. As shown herein, retinal detachment leads to inflammasome activation and that K9 (3Et-3TC: 4-(diethylamino)-1-[2-(ethoxy methyl)-1,3-oxathiolan-5-yl]-1,2-dihydropyrimidin-2-one (also referred to herein as Kamuvudine 9), a drug that blocks inflammasome activation, as well as other drugs disclosed herein, prevents photoreceptor cell death and preserves and improves retinal function, as measured by electroretinography (ERG). K9, and similar inflammasome inhibitors, can be used for neuroprotection of photoreceptors not only in retinal detachment but many other retinal degenerations and dystrophies, in which photoreceptors undergo cell death. In addition, they can be used for protection of neurons in the central nervous system and the peripheral nervous system in numerous neurological disorders driven by inflammasome activation.

One candidate pathway is the NLRP3 inflammasome, a multimeric protein complex that recognizes damage-associated molecular patterns (DAMPs) released by injured photoreceptors and launches a potent inflammatory response characterized by caspase-1 activation, interleukin-1β (IL-1β) release, and cell death. (10) Previously, it was discovered that NRTIs, which are used to treat HIV and Hepatitis B infections, also inhibit inflammasome activation independent of their antiretroviral activity. (11) Previous studies demonstrated that NRTIs block inflammasome activation and its associated damage in a variety of contexts, including models of diabetes, (12) diabetic retinopathy, (13,14) aging, (15) choroidal neovascularization, (16) and atrophic age-related macular degeneration. (11,17-19) Therefore, it was believed that the anti-inflammatory effects of NRTIs might also be protective in RRD. In addition to NRTIs, which have numerous adverse effects owing to their off-target effects on host polymerases (20), Kamuvudine-9 (K-9), an NRTI-derivative that has been engineered to avoid undesirable host polymerase inhibition yet retain inflammasome suppression (11,18,21), was tested.

A mouse model of RRD was employed in which a retinal tear is created and a viscous substance, carboxymethylcellulose (CMC), is injected into the subretinal space, thereby simulating human RRD in which the vitreous humor migrates into the subretinal space via the retinal tear created by vitreoretinal traction. In the standard RRD model, the retina remains detached for weeks, often months, during which time electroretinography (ERG) is nearly non-recordable. (22) Thus, the ability of an intervention to protect or improve electrical function cannot be assessed. Therefore, a new mouse model of RRD was developed with spontaneous retinal reattachment (SRR) to better simulate the clinical course of RRD and to enable evaluation of K-9 treatment on functional outcomes following retinal reattachment.

One aspect provides a method to treat rhegmatogenous retinal detachment (RRD) and/or improve visual outcomes of RRD comprising administering to a subject in need thereof one or more inflammasome inhibitors. Another aspect provides a method for neuroprotection of photoreceptors comprising administering to a subject with retinal detachment one or more inflammasome inhibitors. One aspect provides a method to inhibit photoreceptor cell death comprising administering to a subject administering with retinal detachment one or more inflammasome inhibitors. Another aspect provides a method to preserve and/or improve retinal function comprising administering to a subject administering with retinal detachment one or more inflammasome inhibitors. In one aspect, retinal function is measured by electroretinography (ERG). In one aspect, the subject has complete or partial retinal detachment. In another aspect, the subject undergoes surgery for retinal detachment after administration of the one or more inflammasome inhibitors. In one aspect, the one or more inflammasome inhibitors are administered orally or injected. In one embodiment, the one or more inflammasome inhibitors are injected into an eye of the subject, such as the vitreous humor of the eye of the subject.

In one embodiment, the one or more inflammasome inhibitors comprises a compound of structural Formula (I)

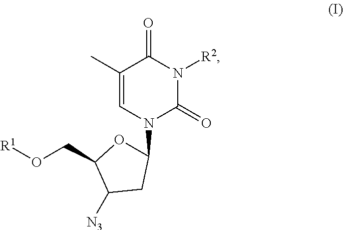

wherein:
R¹ is C₁₋₄ alkyl; and
R² is H or C₁₋₄ alkyl, provided that when R² is H, R¹ is not CH₃ or a salt thereof. In one embodiment, R² is CH₃ or CH₂CH₃. In another embodiment, R¹ is n-C₄H₉.

In one embodiment, the one or more inflammasome inhibitors comprises one or more of

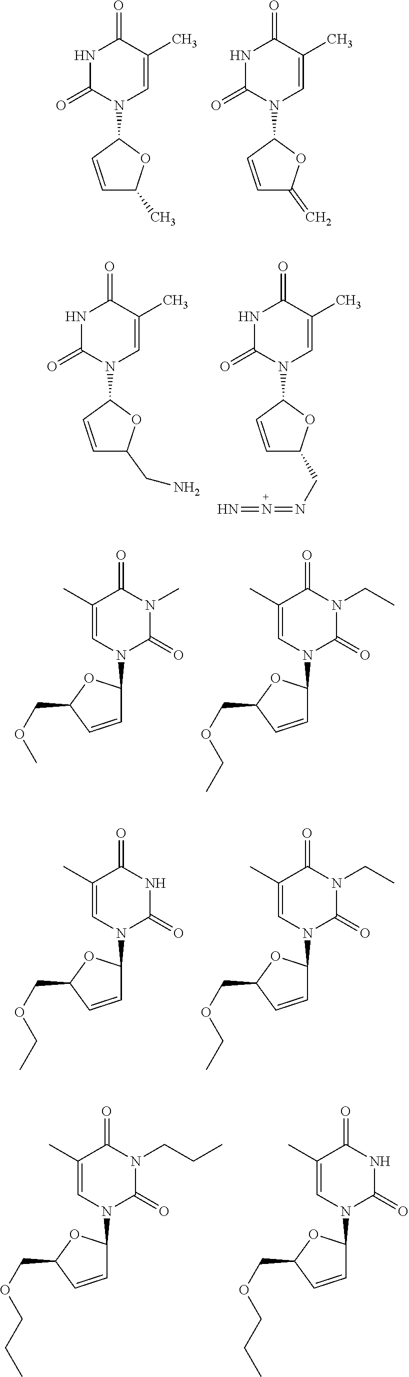

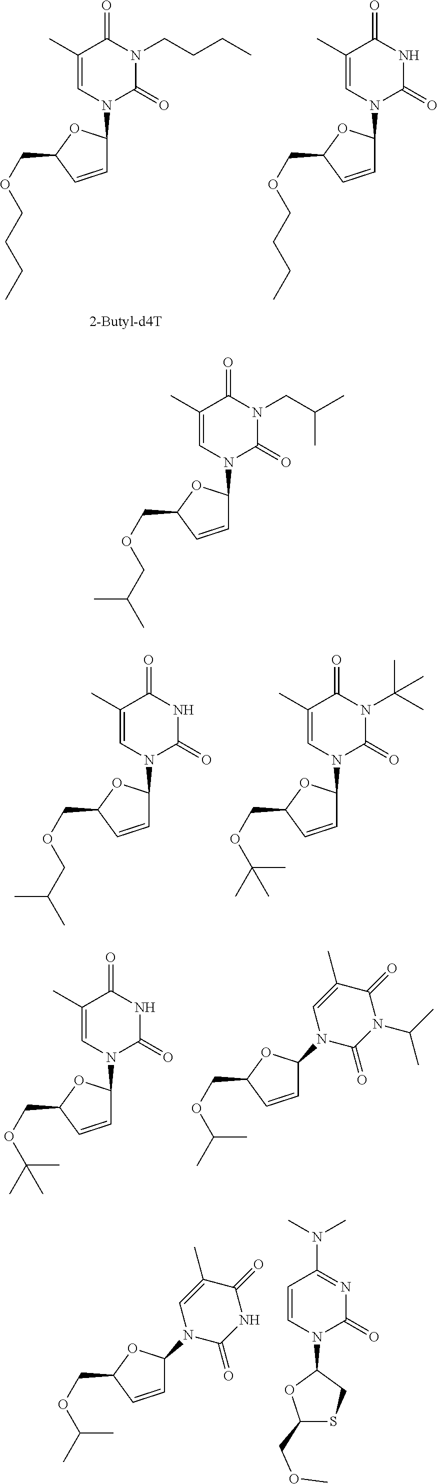

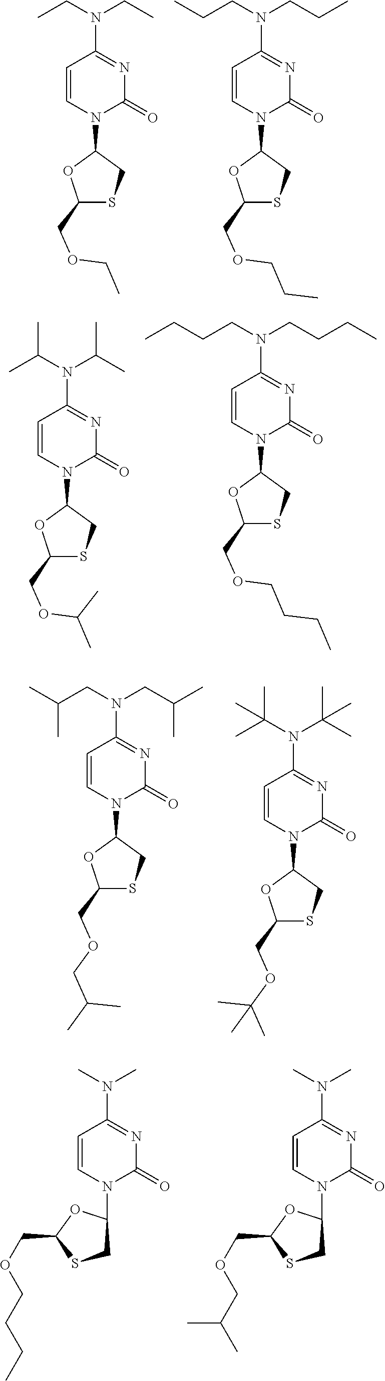

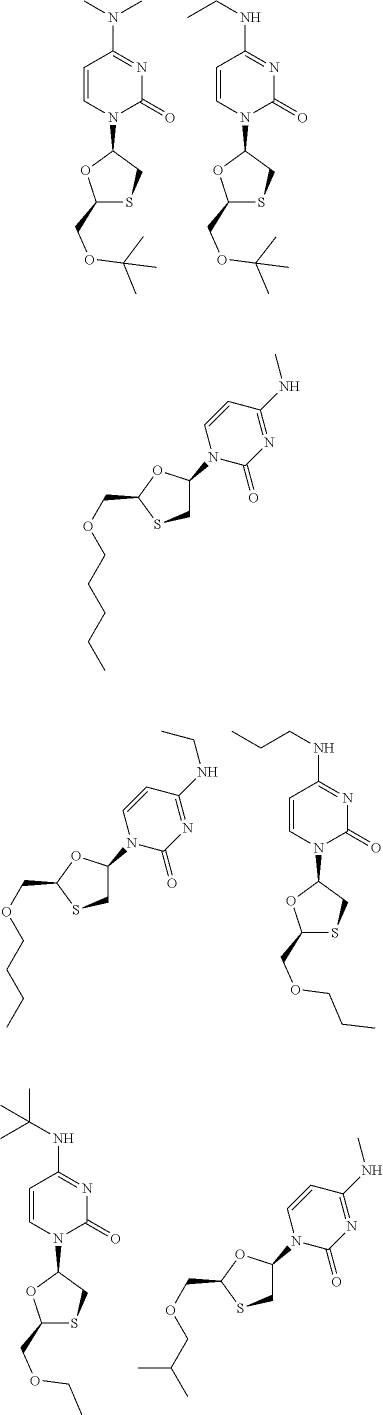

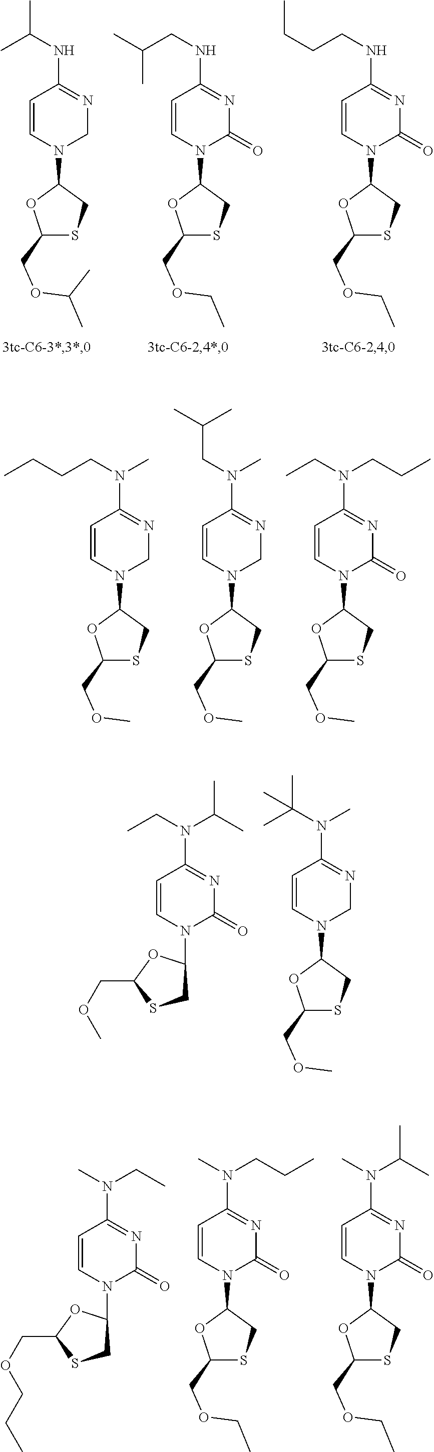

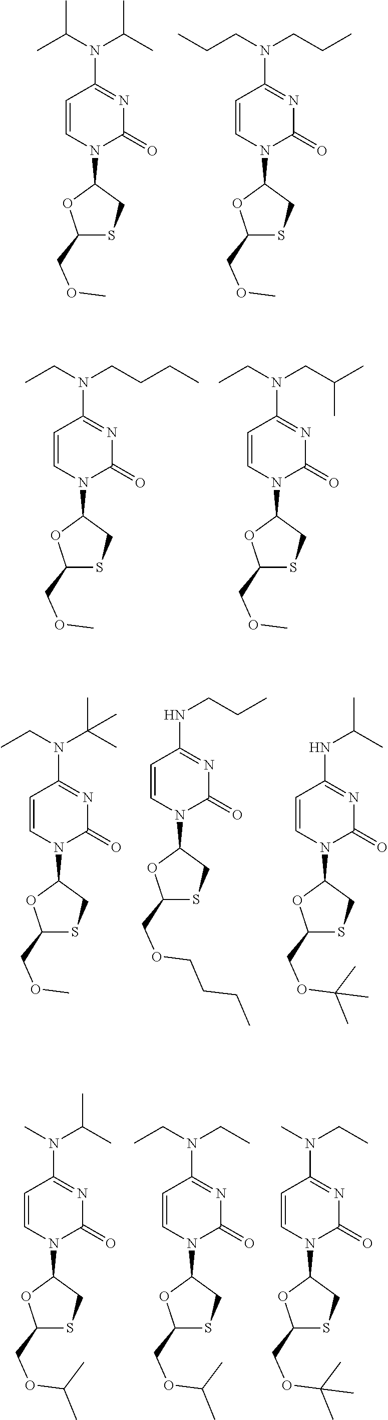

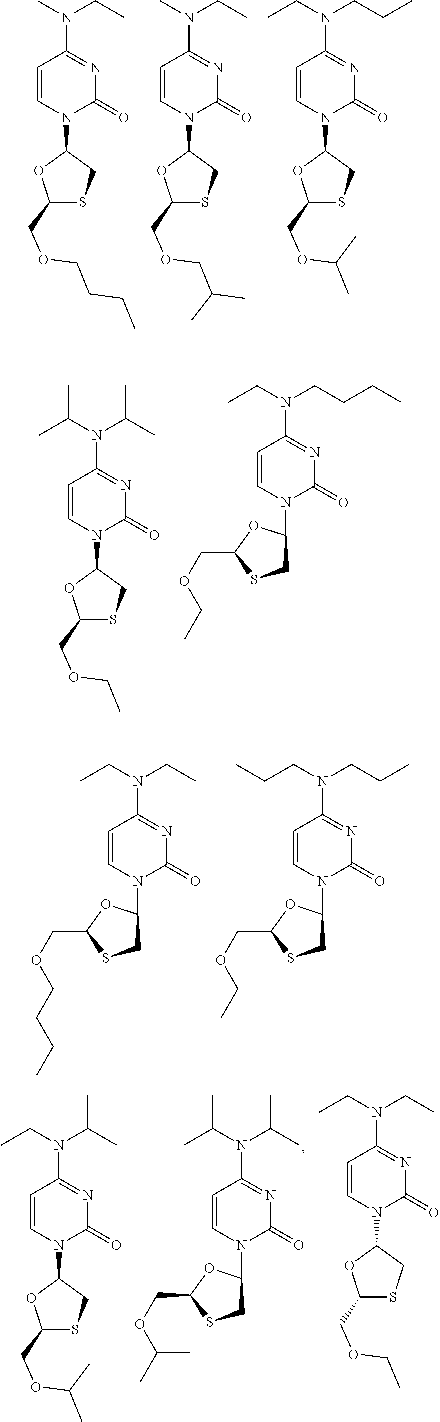

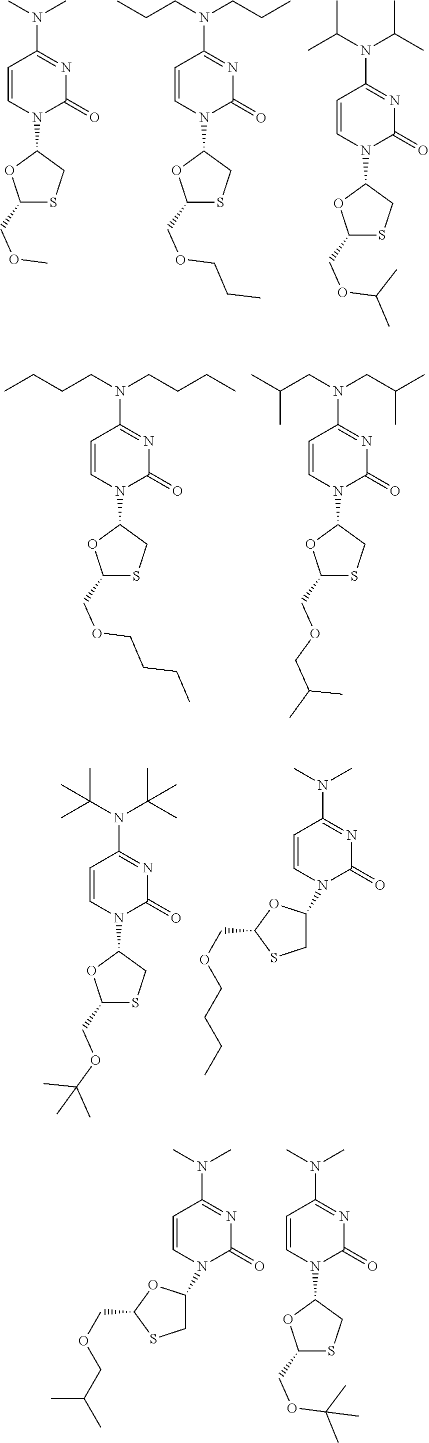

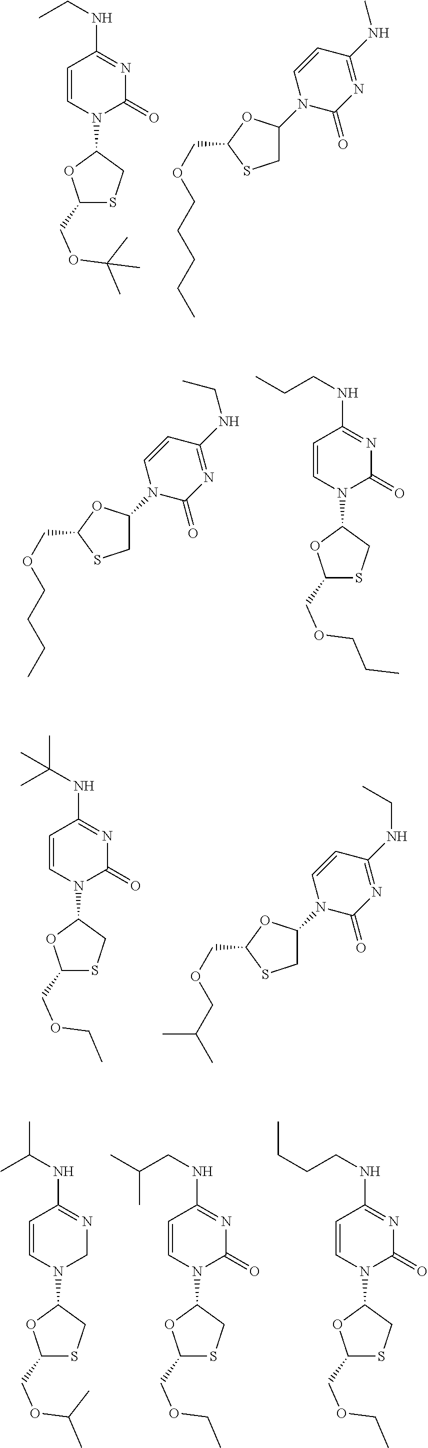

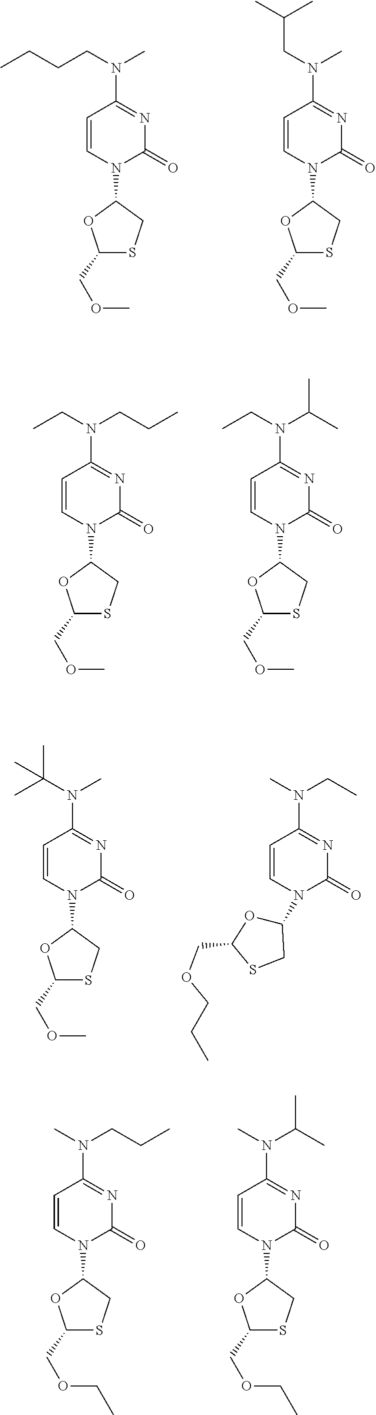

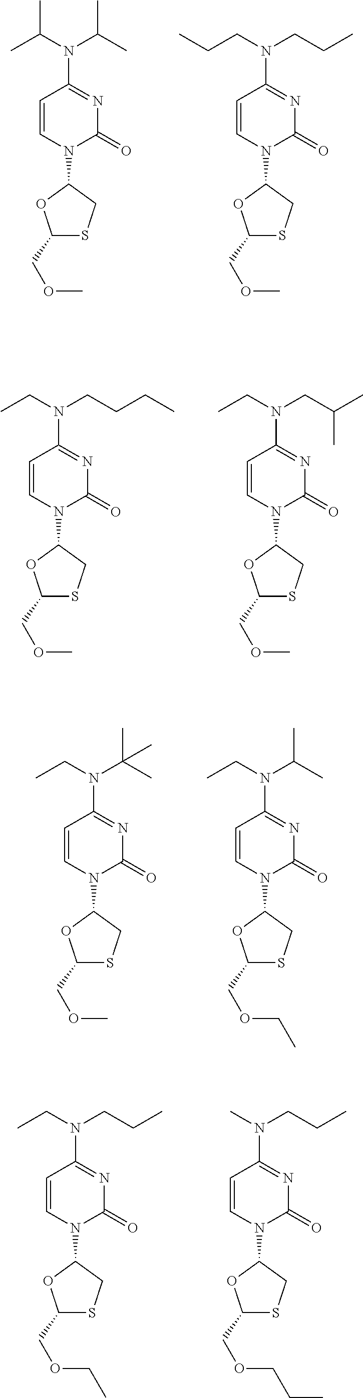

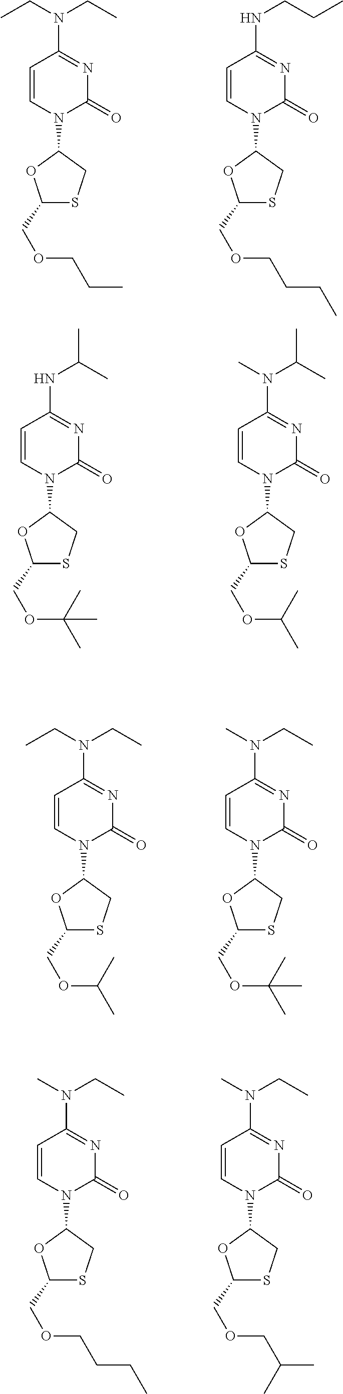

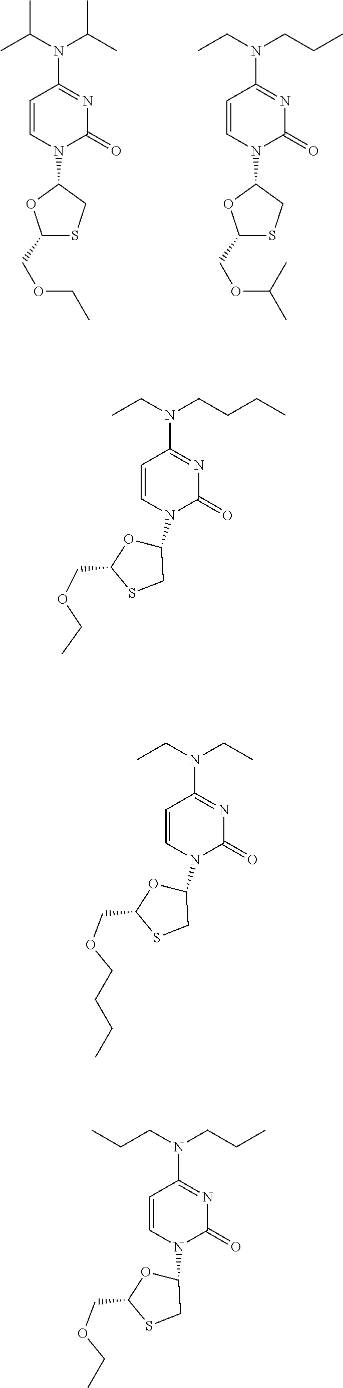 [SMILES: CCOC[C@@H]1O[C@H](n2ccc(N(C(C)C)C(C)C)nc2=O)CS1 ; CCCN(CC)c1ccn([C@@H]2CS[C@H](COC(C)C)O2)c(=O)n1 ; CCCCN(CC)c1ccn([C@@H]2CS[C@H](COCC)O2)c(=O)n1 ; CCCCOC[C@@H]1O[C@H](n2ccc(N(CC)CC)nc2=O)CS1 ; CCCN(CCC)c1ccn([C@@H]2CS[C@H](COCC)O2)c(=O)n1]

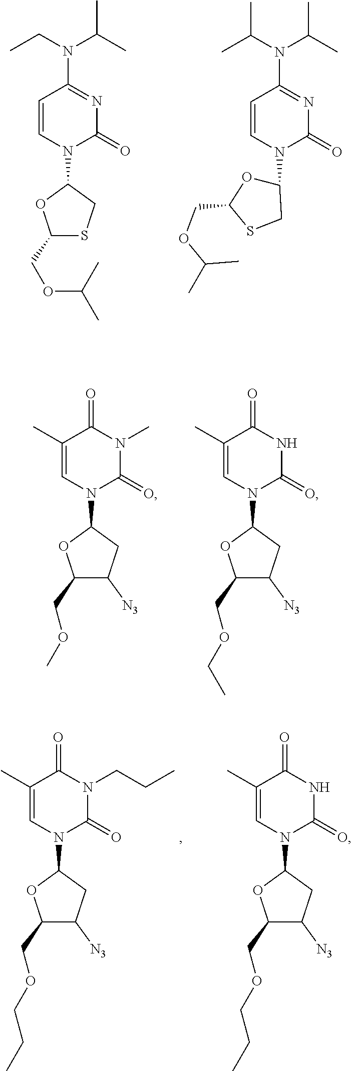

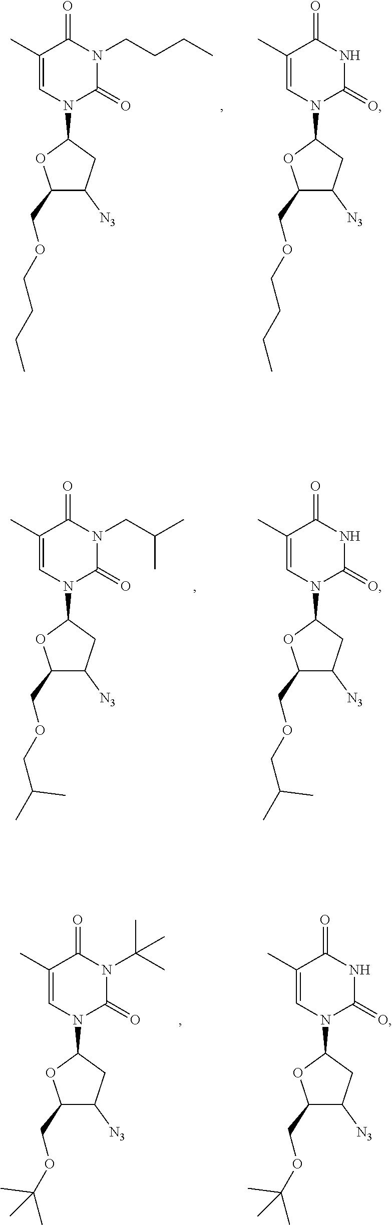 [SMILES: CCCCOC[C@H]1O[C@@H](n2cc(C)c(=O)n(CCCC)c2=O)CC1N=[N+]=[N-] ; CCCCOC[C@H]1O[C@@H](n2cc(C)c(=O)[nH]c2=O)CC1N=[N+]=[N-] ; Cc1cn([C@H]2CC(N=[N+]=[N-])[C@@H](COCC(C)C)O2)c(=O)n(CC(C)C)c1=O ; Cc1cn([C@H]2CC(N=[N+]=[N-])[C@@H](COCC(C)C)O2)c(=O)[nH]c1=O ; Cc1cn([C@H]2CC(N=[N+]=[N-])[C@@H](COC(C)(C)C)O2)c(=O)n(C(C)(C)C)c1=O ; Cc1cn([C@H]2CC(N=[N+]=[N-])[C@@H](COC(C)(C)C)O2)c(=O)[nH]c1=O]

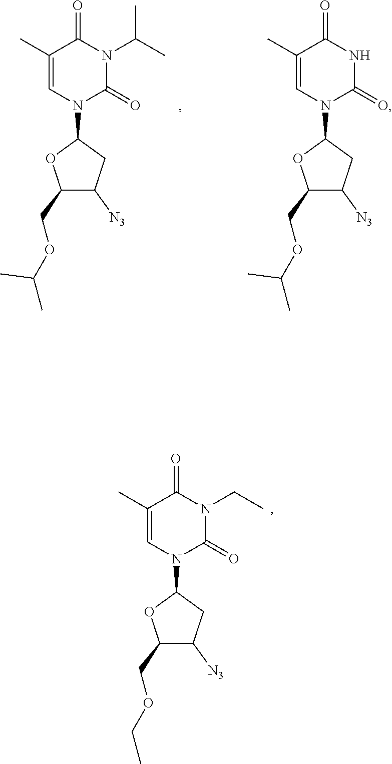 [SMILES: Cc1cn([C@H]2CC(N=[N+]=[N-])[C@@H](COC(C)C)O2)c(=O)n(C(C)C)c1=O ; Cc1cn([C@H]2CC(N=[N+]=[N-])[C@@H](COC(C)C)O2)c(=O)[nH]c1=O ; CCOC[C@H]1O[C@@H](n2cc(C)c(=O)n(CC)c2=O)CC1N=[N+]=[N-]]

an enantiomer or a pharmaceutically acceptable salt thereof.

In one embodiment, the one or more inflammasome inhibitors are Kamuvudine-9 (K-9), lamivudine (3TC), azidothymidine (AZT), a salt thereof or a combination thereof. In another embodiment, the one or more inflammasome inhibitors are

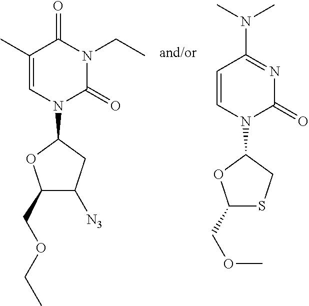 [SMILES: CCOC[C@H]1O[C@@H](n2cc(C)c(=O)n(CC)c2=O)CC1N=[N+]=[N-] ; COC[C@@H]1O[C@H](n2ccc(N(C)C)nc2=O)CS1]

an enantiomer or pharmaceutically acceptable salt thereof.

One embodiment provides a method of making a mouse model of RRD comprising subretinal injection (SRI) of about 1 μl to about 5 μl of about 0.01% to about 0.05% carboxymethyl cellulose (CMC), so as to result in the mouse model of RRD that achieves spontaneous retinal reattachment after 5 to 10 days of RRD. In one embodiment, 3 μl of 0.03% CMC is injected into the mouse.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A-1D. K-9 inhibits RRD-induced caspase-1 cleavage. (A) Representative immunoblot; retinal pro-caspase-1 and caspase-1 in RRD model at baseline, 24-hrs, 48-hrs, and 72-hrs post-SRI (3 μl 1% CMC; WT mice; n=3). (B) Densitometric quantification of caspase-1 from FIG. 1A, normalized to GAPDH (two-tailed t-test; *P<0.05; **P<0.01; n=3; shown as mean±SEM). (C) Representative immunoblot; day 3 retinal pro-caspase-1 and caspase-1 in RRD model treated with PBS, K-9L, or K-9H (n=3). (D) Densitometric quantification of caspase-1 from FIG. 1C, normalized to GAPDH (two-tailed t-test; *P<0.05; ***P<0.001, n=3; shown as mean±SEM).

FIGS. 2A-2B. NRTIs inhibit RRD-induced caspase-1 cleavage (A) Representative immunoblot; day 3 retinal pro-caspase-1 and caspase-1 in RRD model after IP PBS, 3TC, or AZT (n=3). (B) Densitometric quantification of caspase-1 from FIG. 2A normalized to GAPDH (Student's two-tailed t-test; *P<0.05; **P<0.01; ***P<0.001; n=3; shown as mean±SEM).

FIGS. 3A-3B. K-9 protects photoreceptors in RRD model (A) Representative confocal photographs of retinal ONL from day 3 RRD models treated with PBS, K-9L, or K-9H (n=12-14 per group); stained with TUNEL (above; green) and TUNEL/DAPI (below; green and blue). Scale bars represent 100 μm. (B) Quantification of TUNEL-positive ONL cells (Student's two-tailed t-test; **P<0.01; ****P<0.0001; n=12-14 per group; data represented as mean±SEM).

FIGS. 4A-4B. NRTIs attenuate photoreceptor death in RRD model (A) Representative confocal photographs of retinal ONL from day-3 RRD models treated with 3TC, AZT, or PBS (n=12-14 per group); stained with TUNEL (above; green) or TUNEL/DAPI (below; green and blue). Scale bars represent 100 μm. (B) Quantification of day-3 TUNEL-positive ONL cells (Student's two-tailed t-test; **P<0.01; ****P<0.0001; n=12-14 per group; data represented as mean±SEM).

FIGS. 5A-5B. SRI concentration and volume curves used to develop the RRD/SRR model (A) Image-guided SD-OCT; representative retinal images of WT mice 3 days post-SRI of 3 μl CMC of various concentrations 0.01-1% (n=3). (B) Image-guided SD-OCT; mice retinas 1-day post-SRI of 0.03% CMC of various volumes 1˜4 μl (n=3). Scale bars represent 100 μm.

FIGS. 6A-6B. Kinetics of retinal attachment in RRD/SRR model (A) Image-guided SD-OCT; representative images of the right (OD) and left (OS) retinas of WT mice over days 0-10 post-SRI of 3 μl 0.03% CMC (n=4). Scale bars represent 100 μm. (B) Incidence of spontaneous retinal reattachment (SRR) over time (n=4).

FIGS. 7A-7D. K-9 improves ERG functional outcomes in RRD/SRR model (A) Experiment overview: the RRD/SRR model was established day 0 (SRI of 3 μl 0.03% CMC). K-9 (60 mg/kg) or PBS was administered days 0-10 twice daily IP. Scotopic ERG was measured at baseline, as well as days 7, 11, and 14. In 7A-D, n=10-12 per group. (B) ERG waveforms representative of the K-9 and PBS groups, at baseline and day 14. (C) Amplitudes of a- and b-waves over time in the K-9 (red) and PBS (blue) groups at the stimulation intensity of 1.0 log cd sec/m2. Days are marked for which AA from baseline is significantly different between groups (*P<0.05; **P<0.01; Student's two-tailed t-test). Data (7C-D) is represented as mean±SEM. (D) AA from baseline for PBS (blue) and K-9 (red) groups. As in 7C, days are marked when AA from baseline is significantly different between groups.

FIG. 8. K-9 protects retinal function. Starting at postnatal day 15 (P15) until P28, rd10 mice were treated with K-9 at 60 mg/kg twice daily via intraperitoneal injection or with an equal volume of vehicle (PBS). Retinal function was monitored with scotopic electroretinography (ERG).

### DETAILED DESCRIPTION OF EXEMPLARY EMBODIMENTS

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “physiologically functional derivative” means any pharmaceutically acceptable derivative of a compound of the present disclosure. For example, an amide or ester of a compound which upon administration to a subject, particularly a mammal, is capable of providing, either directly or indirectly, a compound of the present disclosure of an active metabolite thereof.

The terms “treatment” or “treating” refer to the medical management of a subject with the intent to cure, ameliorate, stabilize, or prevent a condition or disorder (e.g., retinal degradation). This term includes active treatment, that is, treatment directed specifically toward the improvement of a condition, and also includes causal treatment, that is, treatment directed toward removal of the cause of the associated condition. In addition, this term includes palliative treatment, that is, treatment designed for the relief of symptoms rather than the curing of the condition; preventative treatment, that is, treatment directed to minimizing or partially or completely inhibiting the development of symptoms or disorders of the associated condition; and supportive treatment, that is, treatment employed to supplement another specific therapy directed toward the improvement of the associated disease, pathological condition, or disorder.

With regard to administering the compound, the term “administering” refers to any method of providing a composition and/or pharmaceutical composition thereof to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, subcutaneous administration, intra vitreous administration, including via intravitreous sustained drug delivery device, intracameral (into anterior chamber) administration, suprachoroidal injection, subretinal administration, subconjunctival injection, sub-Tenon's administration, peribulbar administration, transscleral drug delivery, administration via topical eye drops, and the like. Administration can be continuous or intermittent. In various aspects, a preparation can be administered therapeutically; that is, administered to treat an existing disease or condition (e.g., exposure to OP compounds). In further various aspects, a preparation can be administered prophylactically; that is, administered for prevention of a disease or condition.

The term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition. For example, for oral administration a dose or doses of up to about 1200 mg per day or up to about 600 mg per day or up to about 300 mg per day or up to about 150 mg per day or up to about 50 mg per day can be administered. For example, for intra-vitreous administration a dose or doses of up to about 1 mg or up to about 0.5 mg or up to about 0.25 mg or up to 0.1 about mg can be administered.

The terms “subject” or “subject in need thereof refer to a target of administration, which optionally displays symptoms related to a particular disease, condition, disorder, or the like. The subject(s) of the herein disclosed methods can be human or non-human (e.g., primate, horse, pig, rabbit, dog, sheep, goat, cow, cat, guinea pig, rodent, and non-mammals). The term “subject” does not denote a particular age or sex. Thus, adult and newborn subjects, as well as fetuses, whether male or female, are intended to be covered. The term “subject” includes human and veterinary subjects.

As will be recognized by one of ordinary skill in the art, the terms “suppression,” “suppressing,” “suppressor,” “inhibition,” “inhibiting” or “inhibitor” do not refer to a complete elimination in all cases. Rather, the skilled artisan will understand that the term “suppressing” or “inhibiting” refers to a reduction or decrease. Such reduction or decrease can be determined relative to a control. In some embodiments, the reduction or decrease relative to a control can be about a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% decrease.

In some exemplary embodiments, the presently disclosed subject matter includes methods for treating retinal detachment. Some methods of the present disclosure comprise administering to a subject in need thereof an effective amount of a composition for treating retinal detachment.

As described herein, the presently disclosed subject matter further includes pharmaceutical compositions comprising the compounds described herein together with a pharmaceutically acceptable carrier.

The term “pharmaceutically acceptable carrier” refers to sterile aqueous or nonaqueous solutions, dispersions, suspensions or emulsions, as well as sterile powders for reconstitution into sterile injectable solutions or dispersions just prior to use. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

Suitable formulations include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The formulations can be presented in unit-dose or multi-dose containers, for example sealed ampoules and vials, and can be stored in a frozen or freeze-dried (lyophilized) condition requiring only the addition of sterile liquid carrier immediately prior to use.

For oral administration, the compositions can take the form of, for example, tablets or capsules prepared by a conventional technique with pharmaceutically acceptable excipients such as binding agents {e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycol late); or wetting agents (e.g., sodium lauryl sulphate). The tablets can be coated by methods known in the art.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of tablets or lozenges formulated in conventional manner.

The compositions can be formulated as eye drops. For example, the pharmaceutically acceptable carrier may comprise saline solution or other substances used to formulate eye drop, optionally with other agents. Thus, eye drop formulations permit for topical administration directly to the eye of a subject.

The compositions can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). The compounds can also be formulated in rectal compositions, creams or lotions, or transdermal patches.

The presently disclosed subject matter further includes a kit that can include a compound or pharmaceutical composition as described herein, packaged together with a device useful for administration of the compound or composition. As will be recognized by those or ordinary skill in the art, the appropriate administration-aiding device will depend on the formulation of the compound or composition that is selected and/or the desired administration site. For example, if the formulation of the compound or composition is appropriate for injection in a subject, the device could be a syringe. For another example, if the desired administration site is cell culture media, the device could be a sterile pipette.

As briefly described above, retinal detachment can be spontaneous, caused by aging or trauma. Rhegmatogenous retinal detachment (RRD) is a vision-threatening event that benefits from surgical intervention. While awaiting surgical reattachment, irreversible hypoxic and inflammatory damage to the retina often occurs. An interim therapy protecting photoreceptors can improve functional outcomes.

The methods provided herein comprise administration of one or more compounds of structural Formula (I)

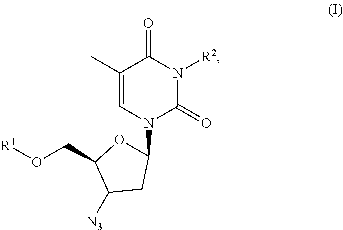

wherein:
R¹ is C₁₋₄ alkyl; and
R² is H or C₁₋₄ alkyl, provided that when R² is H, R¹ is not CH₃ or a pharmaceutically salt thereof and optionally present in a carrier. In some embodiments, R² is CH₃ or CH₂CH₃. In other embodiments, R¹ is n-C₄H₉.

As used herein the term “alkyl” refers to C₁₋₄ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, and/or tert-butyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₄ straight-chain alkyls. In other embodiments, “alkyl” refers, in particular, to C₁₋₄ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The methods provided herein also comprise administration of one or more compounds including:

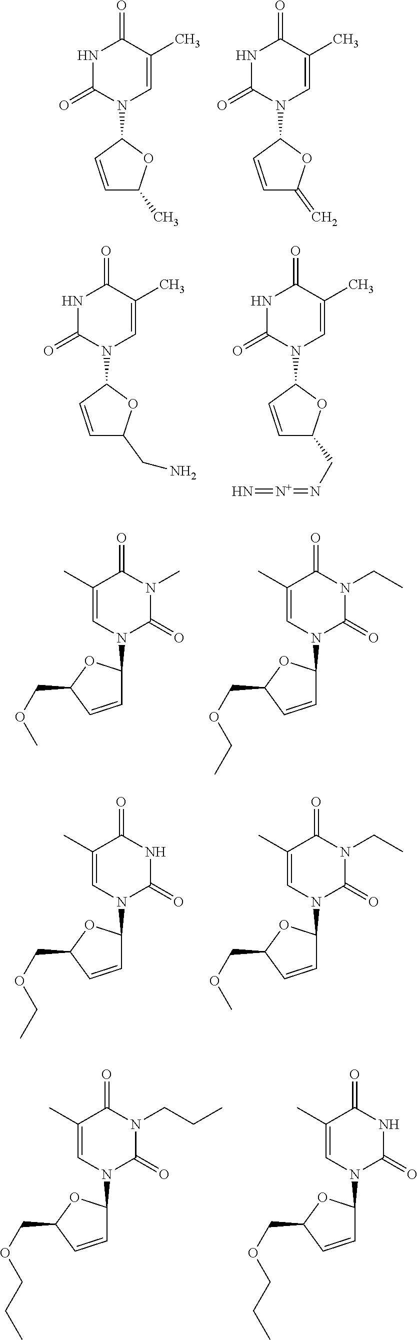

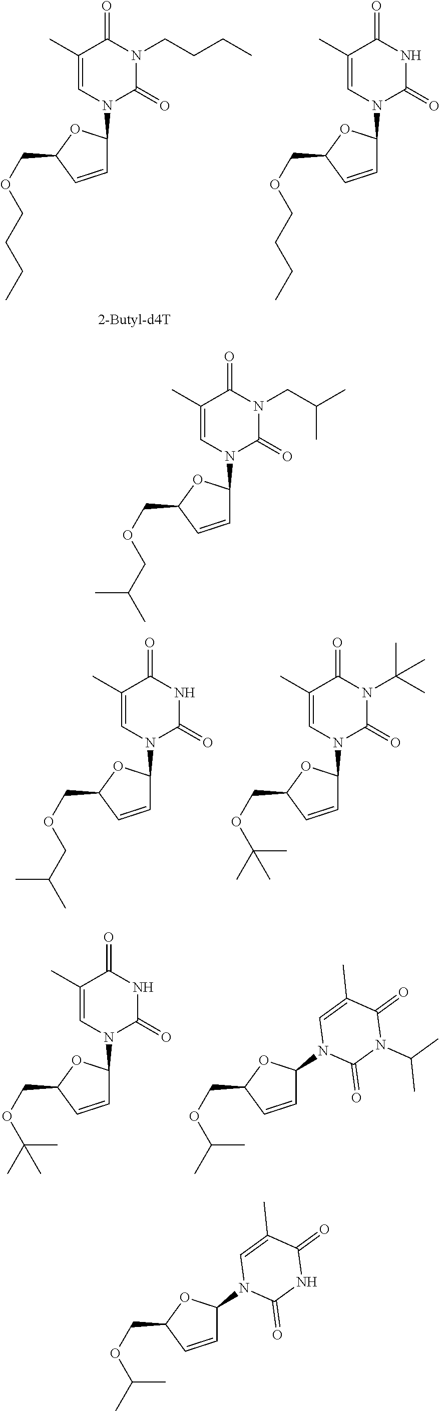

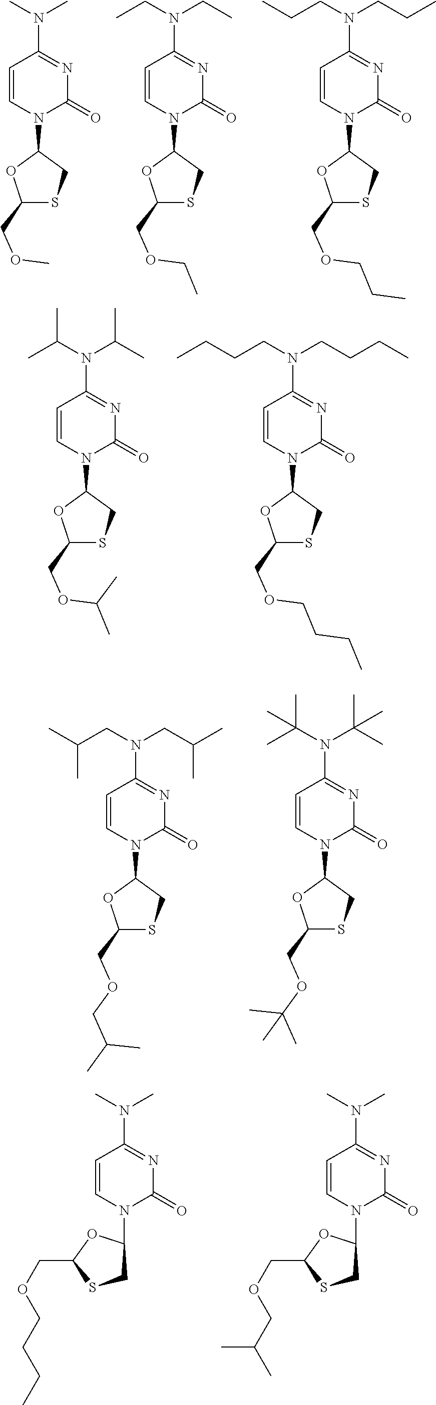

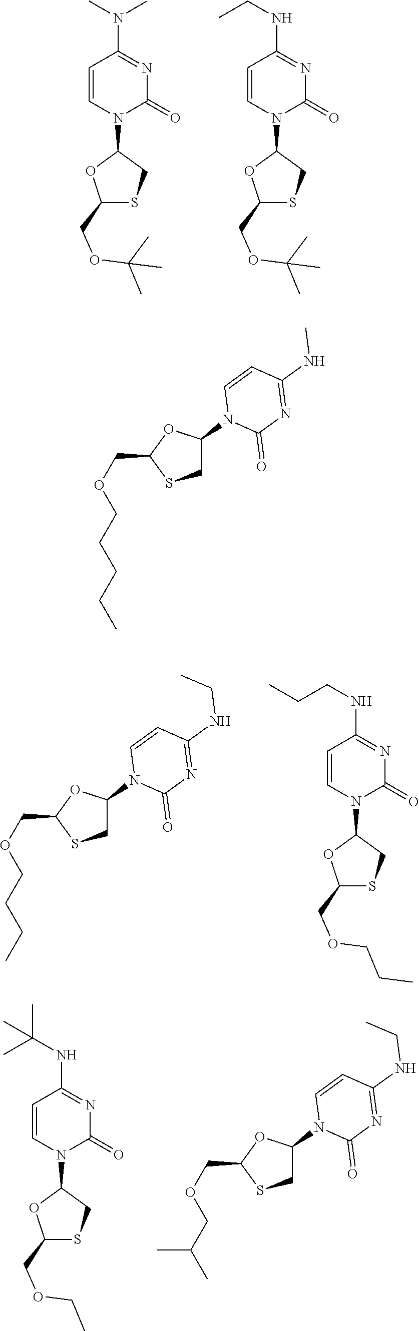

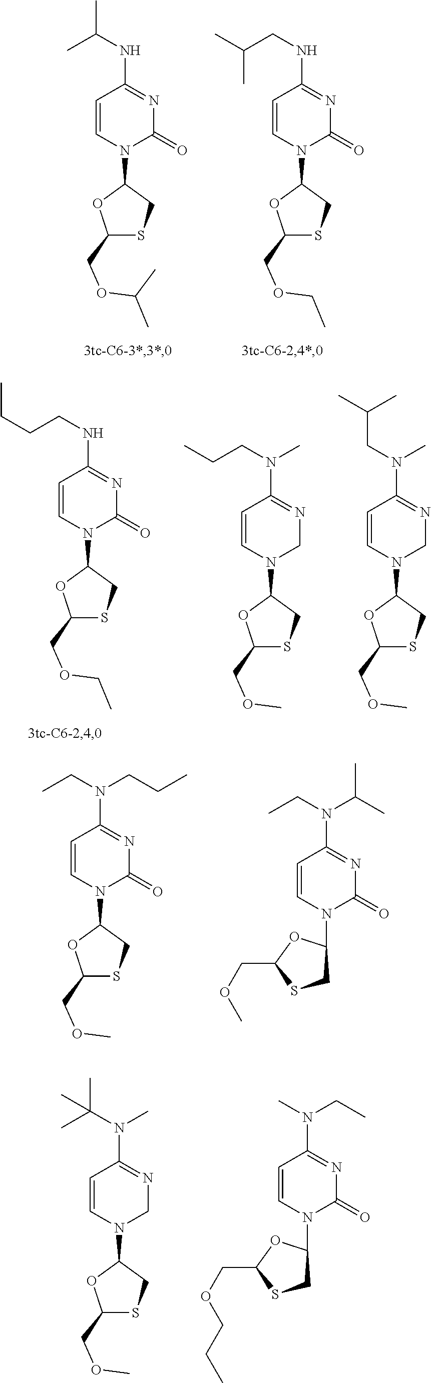

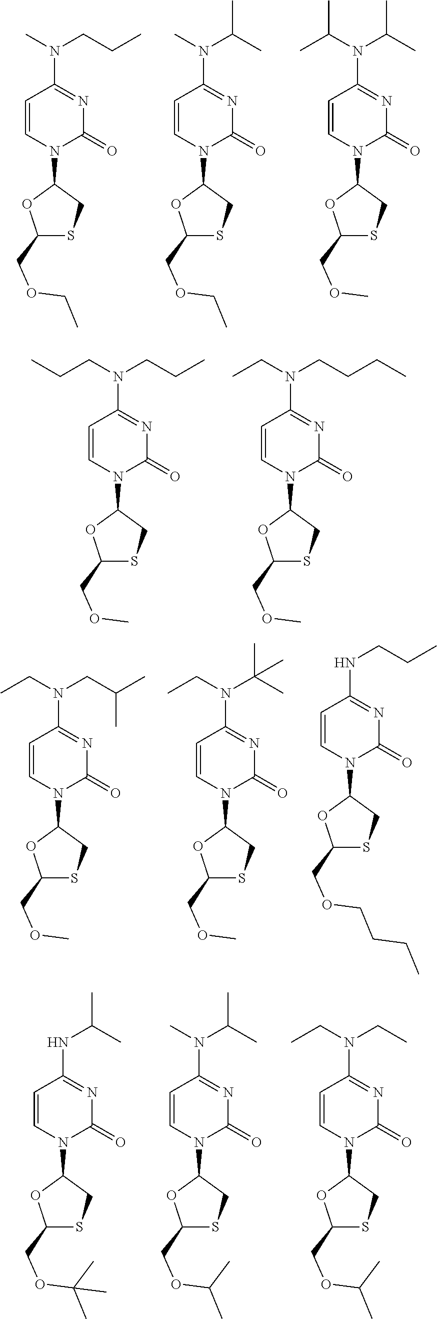

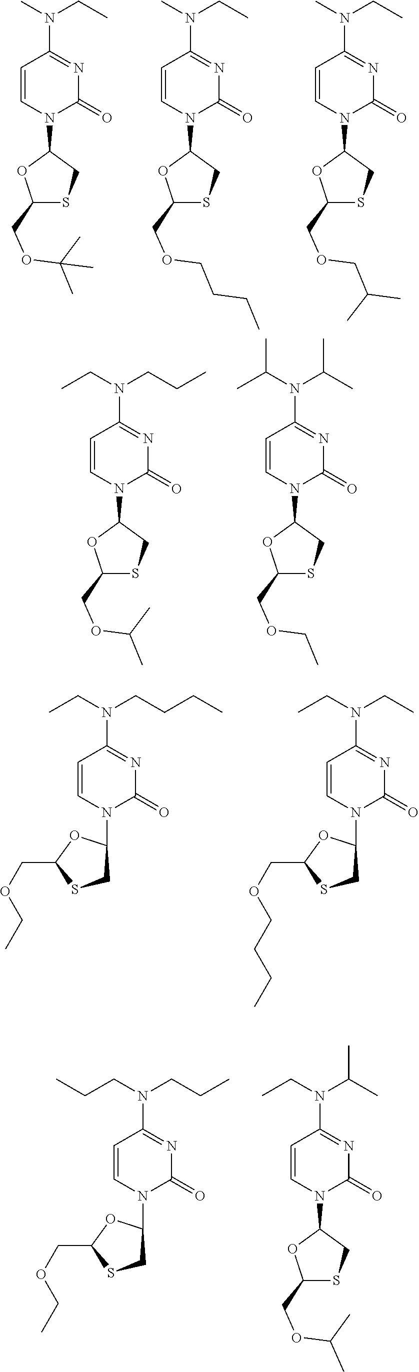

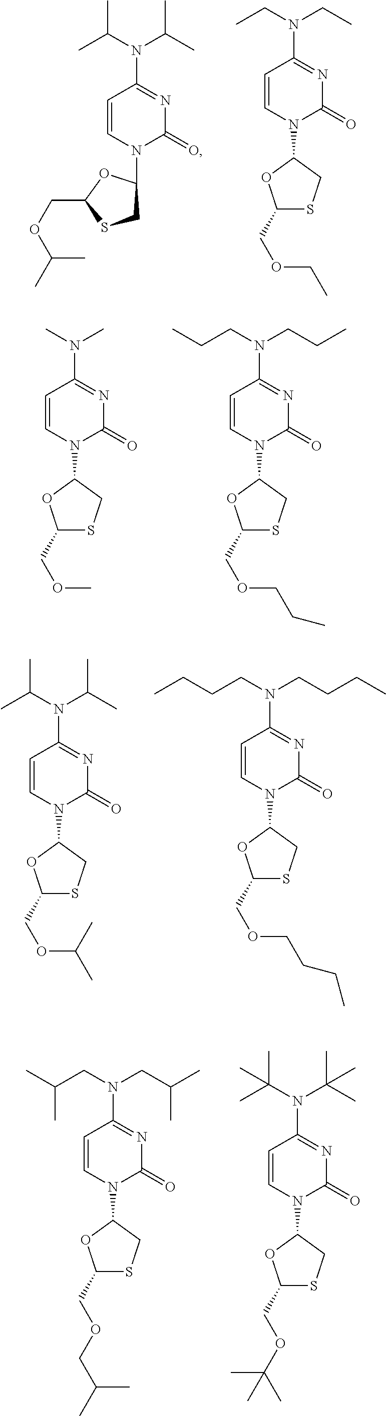

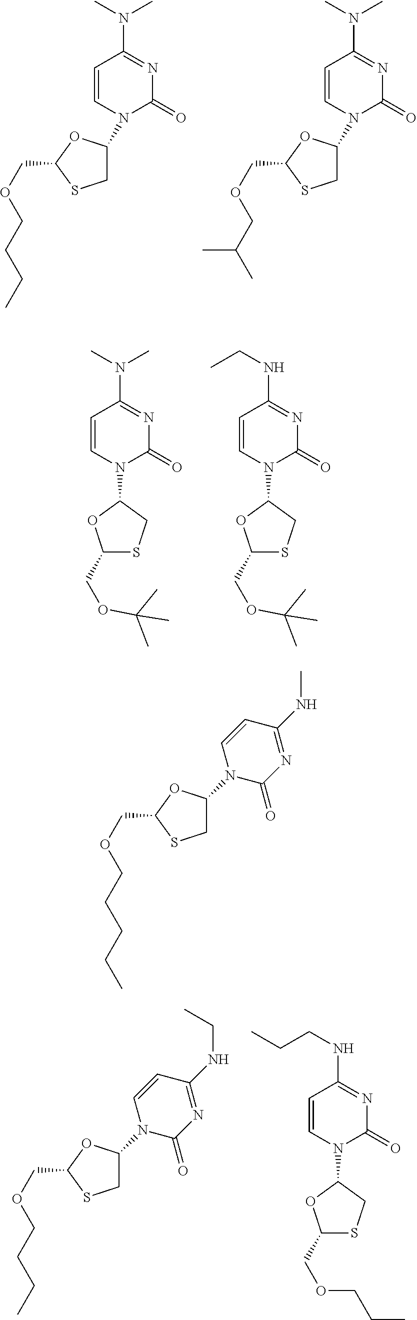

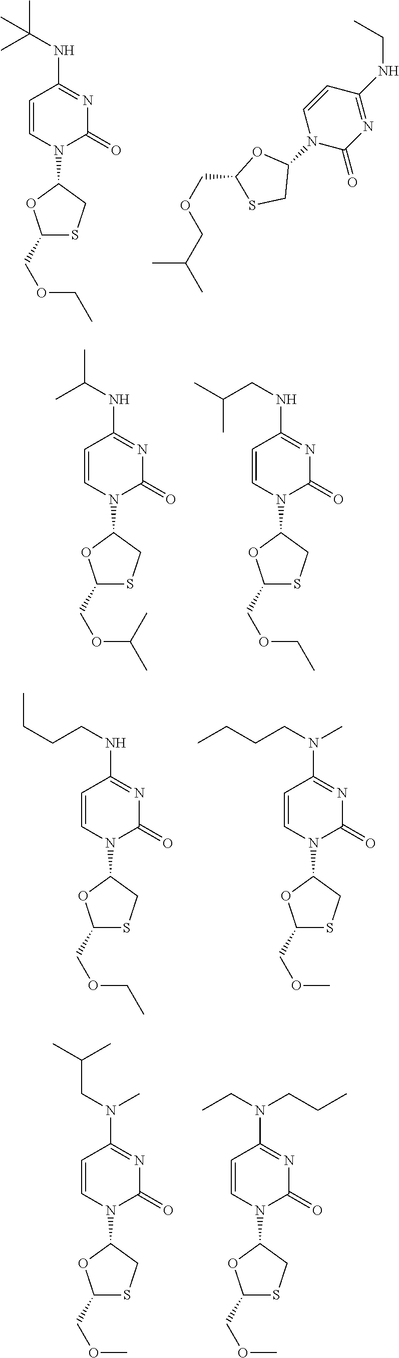

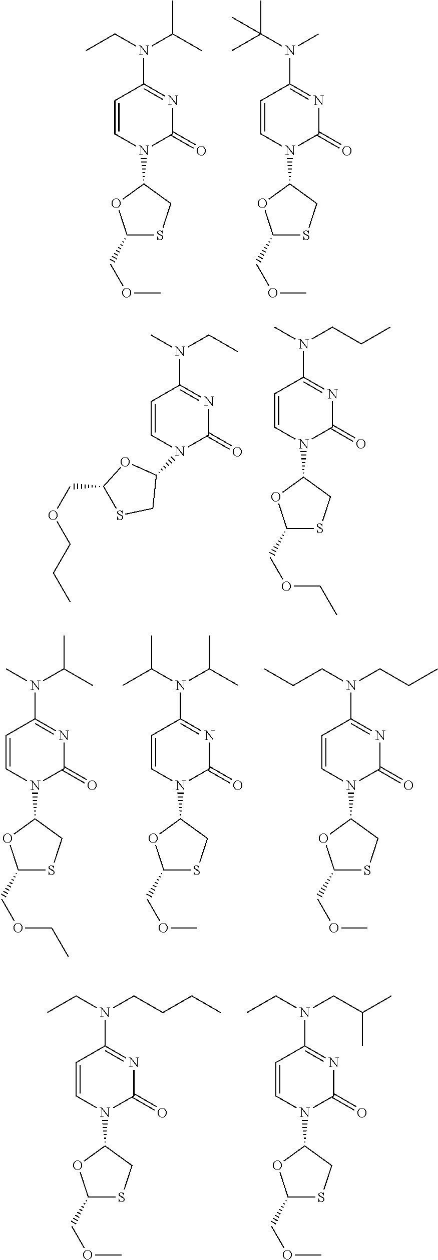

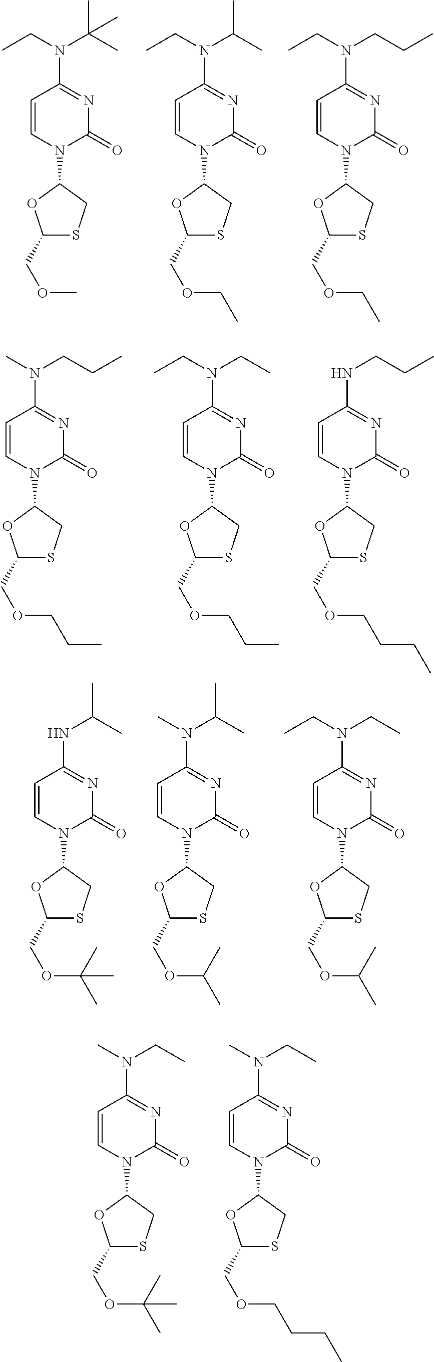

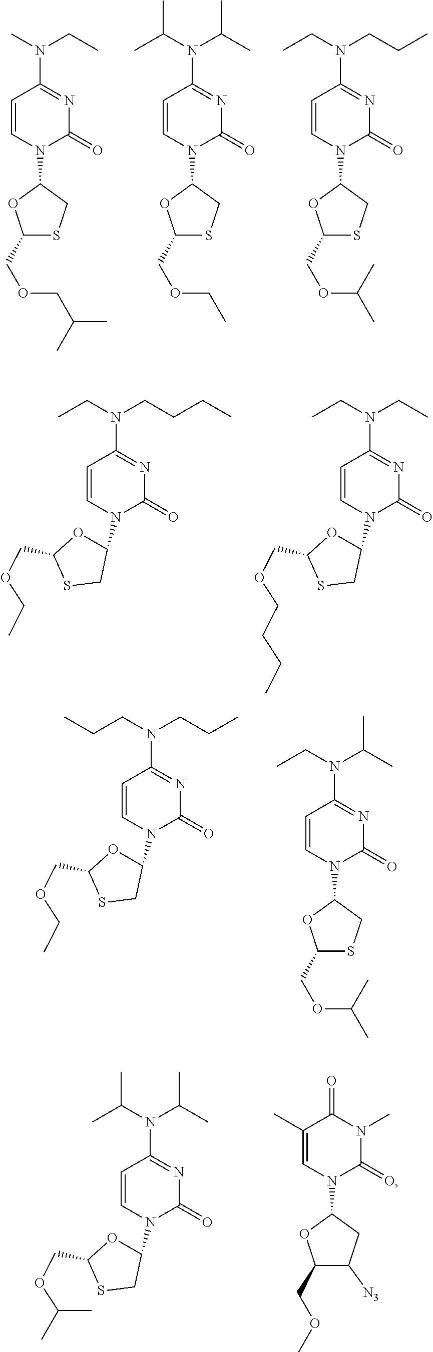

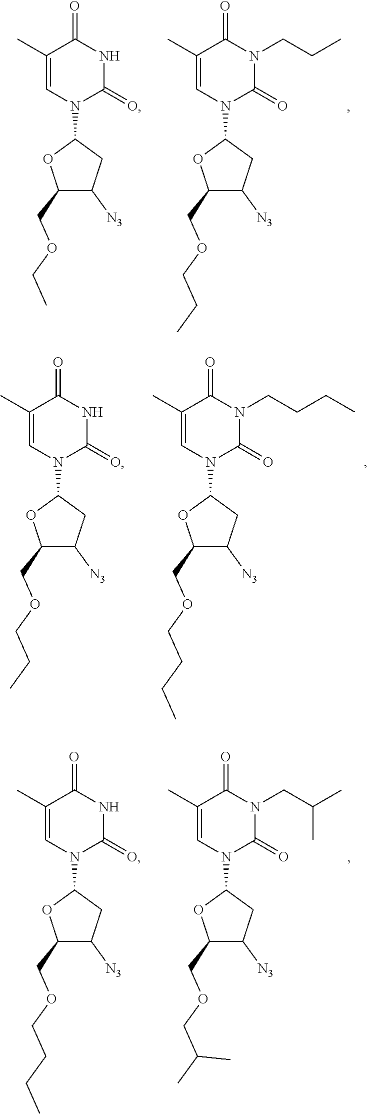

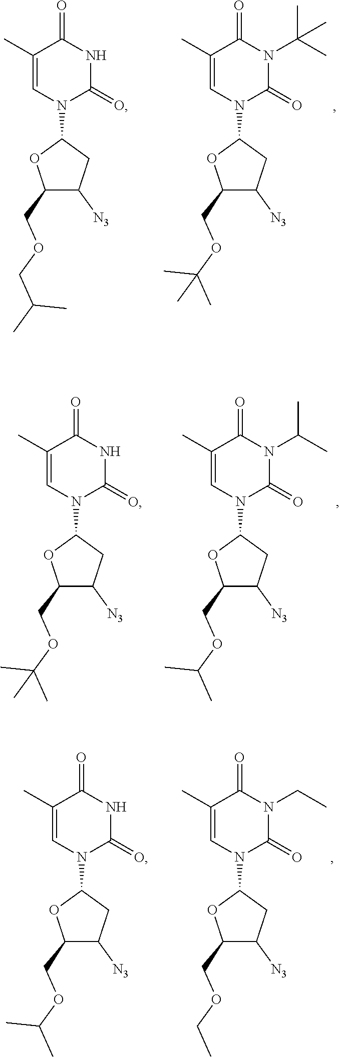 [SMILES: Cc1cn([C@@H]2CC(N=[N+]=[N-])[C@@H](COCC(C)C)O2)c(=O)[nH]c1=O ; Cc1cn([C@@H]2CC(N=[N+]=[N-])[C@@H](COC(C)(C)C)O2)c(=O)n(C(C)(C)C)c1=O ; Cc1cn([C@@H]2CC(N=[N+]=[N-])[C@@H](COC(C)(C)C)O2)c(=O)[nH]c1=O ; Cc1cn([C@@H]2CC(N=[N+]=[N-])[C@@H](COC(C)C)O2)c(=O)n(C(C)C)c1=O ; Cc1cn([C@@H]2CC(N=[N+]=[N-])[C@@H](COC(C)C)O2)c(=O)[nH]c1=O ; CCOC[C@H]1O[C@H](n2cc(C)c(=O)n(CC)c2=O)CC1N=[N+]=[N-]]

an enantiomer or a pharmaceutically acceptable salt thereof.

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently disclosed subject matter is further illustrated by the following specific but non-limiting examples.

### Example I—Kamuvudine-9 Protects Retinal Structure and Function in a Novel Model of Experimental Rhegmatogenous Retinal Detachment

### Introduction

Rhegmatogenous retinal detachment (RRD) is a vision-threatening event that benefits from surgical intervention. While awaiting surgical reattachment, irreversible hypoxic and inflammatory damage to the retina often occurs. An interim therapy protecting photoreceptors can improve functional outcomes. It is shown herein that Kamuvudine-9 (K-9), a derivative of nucleoside reverse transcriptase inhibitors (NRTIs) that inhibits inflammasome activation, and the NRTIs lamivudine (3TC; 2′3′ dideoxycytidine; Zeffix, GlaxoSmithKline)) and azidothymidine (AZT 3-azido-2′,3′-dideoxythymidine; Retrovir, ViiV Healthcare), can protect the retina following RRD.

### Materials and Methods

RRD was induced in mice via subretinal injection (SRI) of 1% carboxymethylcellulose (CMC). To simulate outcomes following the clinical management of RRD, optimal conditions were determined in which SRI of CMC induced spontaneous retinal reattachment (SRR) over 10 days (RRD/SRR). K-9, 3TC, or AZT was administered via intraperitoneal (IP) injection. Inflammasome activation was monitored by cleaved caspase-1 abundance, and photoreceptor death was assessed by TUNEL staining. Retinal function was assessed by full-field scotopic electroretinography (ERG).

### Mice

All experiments involving animals were approved by the University of Virginia Institutional Animal Care and Use Committee (IACUC) and adhered to the Association for Research in Vision and Ophthalmology (ARVO) Statement for the Use of Animals in Ophthalmic and Vision Research. Animal subjects were male and female mice aged 6-10 weeks. Wild type C57BL/6J mice were obtained from the Jackson Laboratory (Bar Harbor, ME, USA). To anesthetize mice for study procedures, ketamine hydrochloride (100 mg/kg; Ft. Dodge Animal Health, Overland Park, KS, USA) and xylazine (10 mg/kg; Phoenix Scientific, St. Joseph, MO, USA) were delivered by intraperitoneal (IP) injection. Pupils were dilated with topical 1% tropicamide and 2.5% phenylephrine (Alcon Laboratories, Ft. Worth, TX, USA).

### RRD Models

A persistent RRD model was established by subretinal injection (SRI) of 3 μl 1% carboxymethyl cellulose (CMC; Refresh Liquigel, Allergan, Irvine, CA, USA). The model of RRD/SRR was achieved by SRI of 3 μl 0.03% CMC. SRIs were performed as previously described (23) using a 35-gauge needle (Ito Co., Fuji, Japan).

### NRTI and Kamuvudine-9 Treatment

NRTIs and K-9 were delivered via intraperitoneal (IP) injection in equimolar amounts. Lamivudine (3TC, 50 mg/kg) or azidothymidine (AZT, 60 mg/kg) was injected twice daily (SelleckChem, Houston, TX, USA). Kamuvudine K-9 (3-methyl-3TC) was synthesized as previously described (21) and administered either at a low dose (K-9L; 90 mg/kg daily) or a high dose (K-9H; 60 mg/kg twice daily, equimolar to NRTI doses). IP injection of phosphate buffered saline (PBS) was used as control.

### Immunoblotting

Retinal tissue was extracted and lysed by sonication in radioimmunoprecipitation assay (RIPA) buffer (MilliporeSigma, Darmstadt, Germany). The Pierce BCA Protein Assay Kit (ThermoFisher Scientific, Waltham, MA, USA) was used to determine protein concentrations of interest. Samples were prepared that contained equal concentrations of total protein (10-100 μg). Protein samples were resolved by SDS-PAGE and transferred onto Immobilon-FL PVDF membranes (Millipore, Billerica, MA, USA). Membranes were blocked in Odyssey Blocking Buffer (PBS) for 1 hour at room temperature, and then incubated overnight with primary antibody at 4° C. Across study experiments, the following antibodies were used: anti-mouse caspase-1 (1:1000; AG-20B-0042; AdipoGen Life Sciences, San Diego, CA, USA), and anti-mouse GAPDH (1:1000; 2118; Cell Signaling, Danvers, MA, USA). To visualize immunoreactive bands, species-specific secondary antibodies with conjugated IRDye were applied (1:2000; LI-COR Biosciences, Lincoln, NE, USA). Immunoblot images were captured using the Odyssey Infrared Imaging System (LI-COR Biosciences) or autoradiography film.

### TUNEL Assay

Retinal cross sections were stained with TUNEL (TdT-mediated dUTP nick-end labeling) using the In Situ Cell Death Detection Kit (Roche Diagnostics, Indianapolis, IN, USA) and visualized using an A1R laser confocal microscope (Nikon, Tokyo, Japan). Fourteen representative sections, including the optic nerve, were used per treatment group. To calculate the number of TUNEL+ cells in the outer nuclear layer (ONL), the retina analysis toolkit of ImageJ (2.1.0; NIH, Bethesda, MD, USA) was used. (24)

### Retinal Imaging

Fundus images were obtained using the TRC-50 IX camera (Topcon, Tokyo, Japan), linked to a digital imaging system (Sony, Tokyo, Japan) or Mm IV Retinal Microscope (Phoenix Research Laboratories, Pleasanton, CA, USA). Spectral Domain Optical Coherence Tomography (SD-OCT) was performed by attaching an OCT2 scan head to the Mm IV Retinal Microscope.

### Electroretinography (ERG)

ERG was recorded at baseline, as well as days 7, 11, and 14 following SRI of CMC. Mice (n=6) were dark adapted overnight, and then ERG was measured using Ganzfeld ERG (Phoenix Laboratories). LabScribe Software (version 3.016800; Phoenix Laboratories) was utilized to collect scotopic combined responses with no background illumination (0 cd/m²). Responses were recorded to white-flash stimuli ranging from −1.7 to 1.0 log cd s/m2. For each stimulus, three responses were obtained and averaged.

### Statistical Analysis

All statistical analyses were conducted using Prism software (version 8.4.0; GraphPad Inc., San Diego, CA, USA). Outcomes were analyzed using a Student's two-tailed t-test, and P values<0.05 were considered statistically significant. Results were reported as the mean±standard error of the mean (SEM).

### Results

RRD induced retinal inflammasome activation and photoreceptor death in mice. Systemic administration of K-9, 3TC, or AZT inhibited retinal inflammasome activation and photoreceptor death. In the RRD/SRR model, K-9 protected retinal electrical function during the time of RRD and induced an improvement following retinal reattachment.

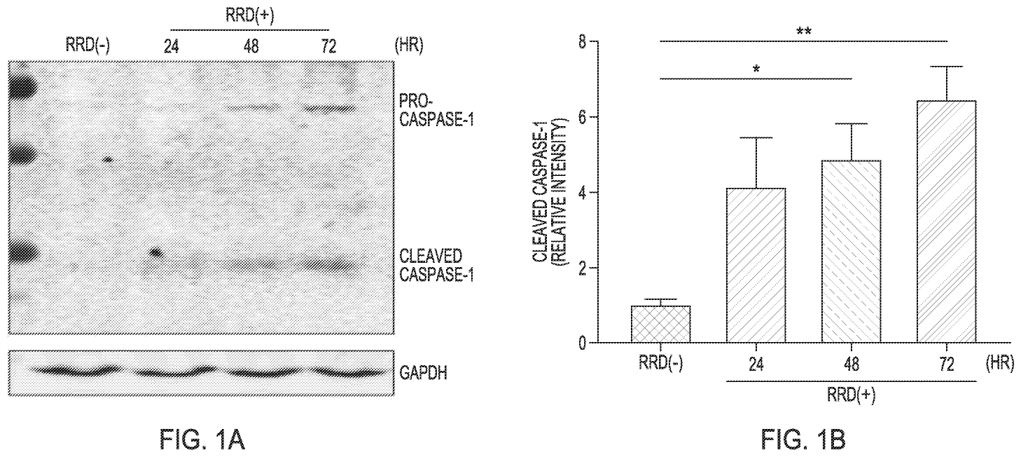

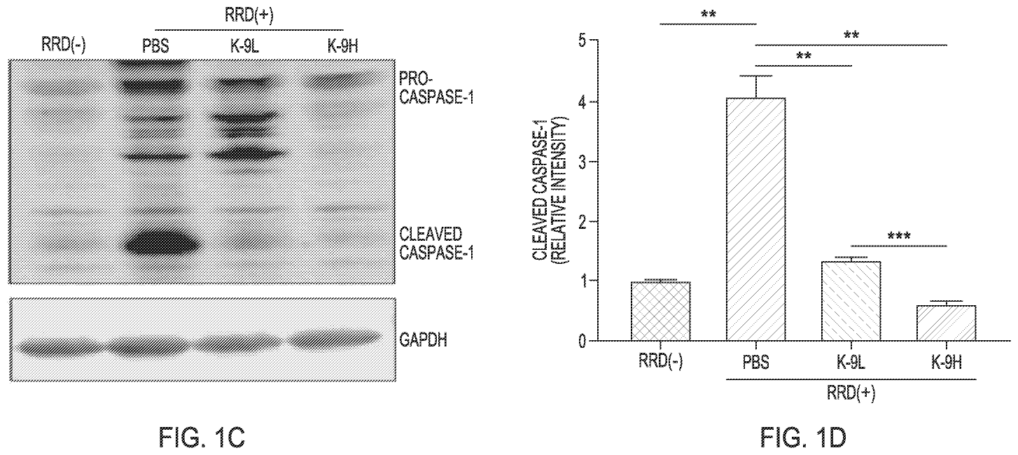

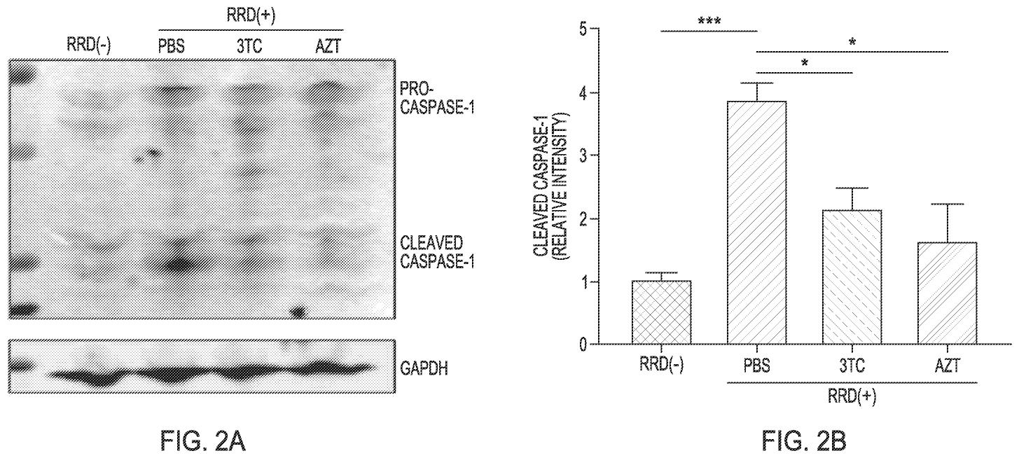

Experimental RRD induced a progressive increase in the abundance of cleaved caspase-1 in retinal lysates isolated from mice over 24-72 hours (FIGS. 1A, 1B). These data indicate in vivo inflammasome activation following RRD and are consistent with hypoxia-induced inflammasome activation. (25,26) Three days after inducing RRD, mice treated with systemic K-9 exhibited dose-dependent and complete reduction in retinal cleaved caspase-1, compared with PBS-treated mice (FIGS. 1C, 1D). Three days after inducing RRD, mice treated with either of the NRTIs (3TC or AZT), exhibited a marked reduction in retinal caspase-1 levels, compared with PBS-treated mice (FIGS. 2A, 2B). These results are compatible with the known inflammasome inhibitory activity of K-9 and NRTIs.11 Of note, K-9 exerted a greater degree of suppression of caspase-1 activation than the NRTIs (84%±1% versus 52%±8%, P<0.05).

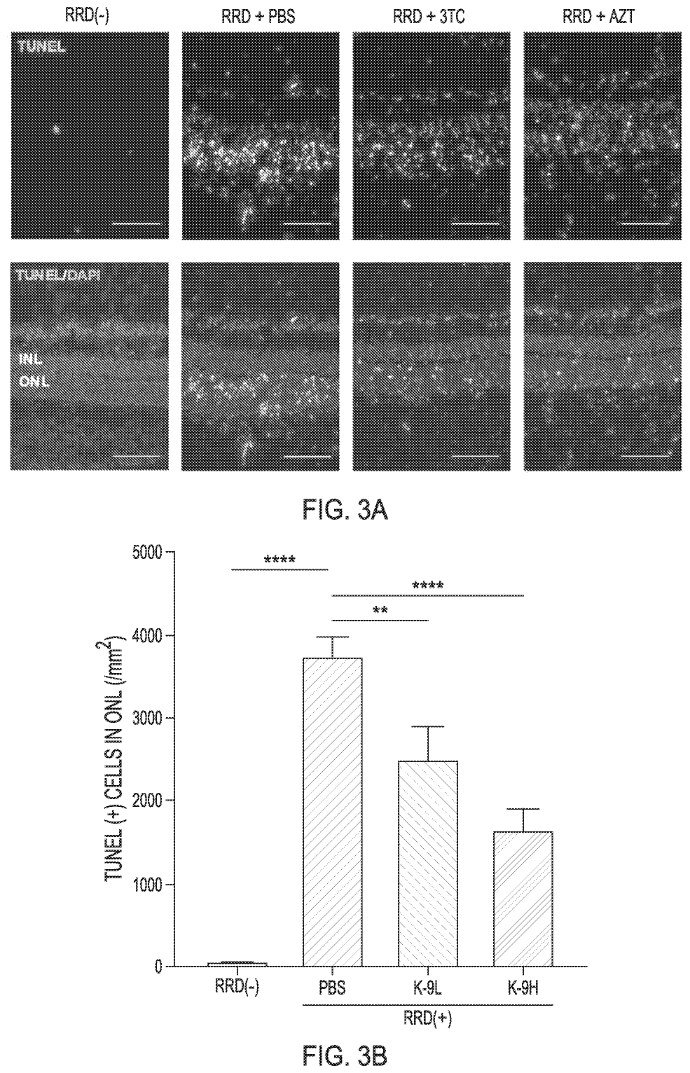

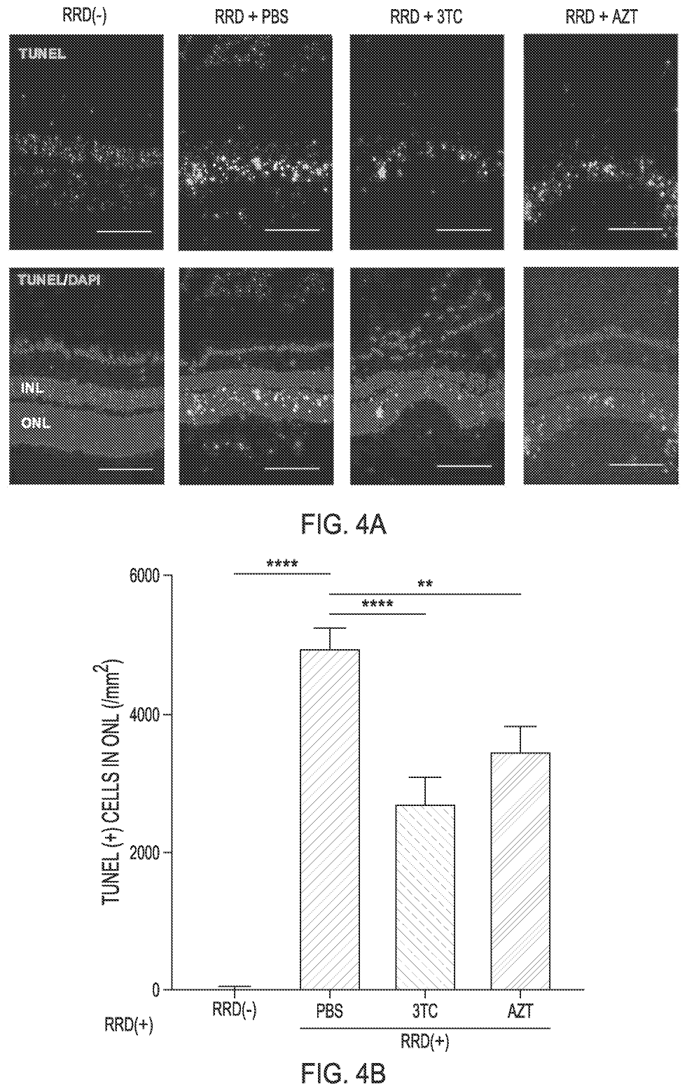

Next, retinal neuroprotection was analyzed by assessing TUNEL staining to measure dead or dying cells. Three days after inducing RRD, a dose-dependent reduction in the number of TUNEL+ cells in the ONL of the retina following K-9 treatment, compared with PBS-treatment (FIG. 3) was observed. Similarly, mice treated with systemic 3TC or AZT displayed significantly fewer TUNEL+ retinal cells, compared with PBS-treated mice (FIG. 4). These findings demonstrate that systemic delivery of these three inflammasome inhibitors can protect photoreceptors from damage during periods of RRD. Of note, K-9 conferred a greater degree of photoreceptor protection than the NRTIs (56%±7% versus 38%±10%, P<0.05).

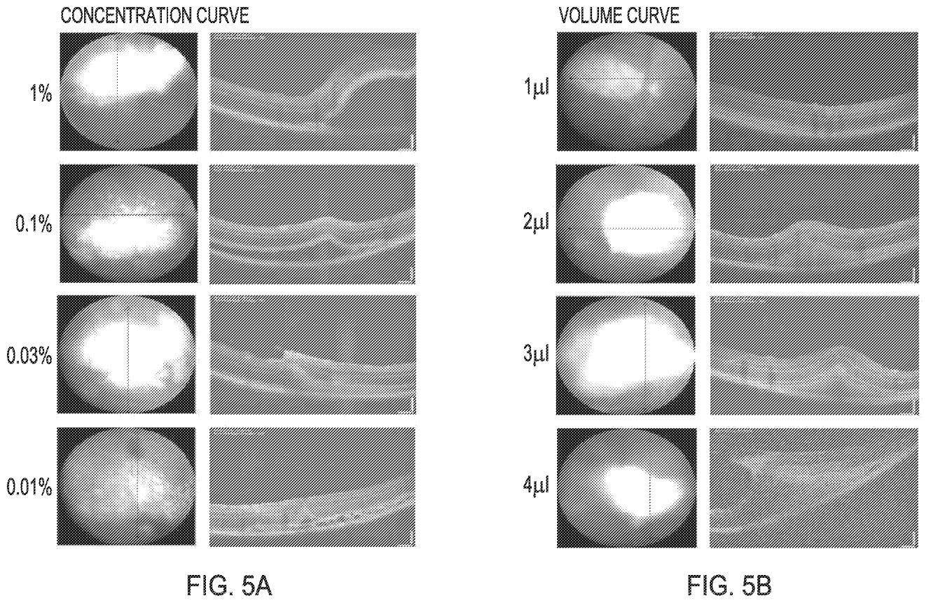

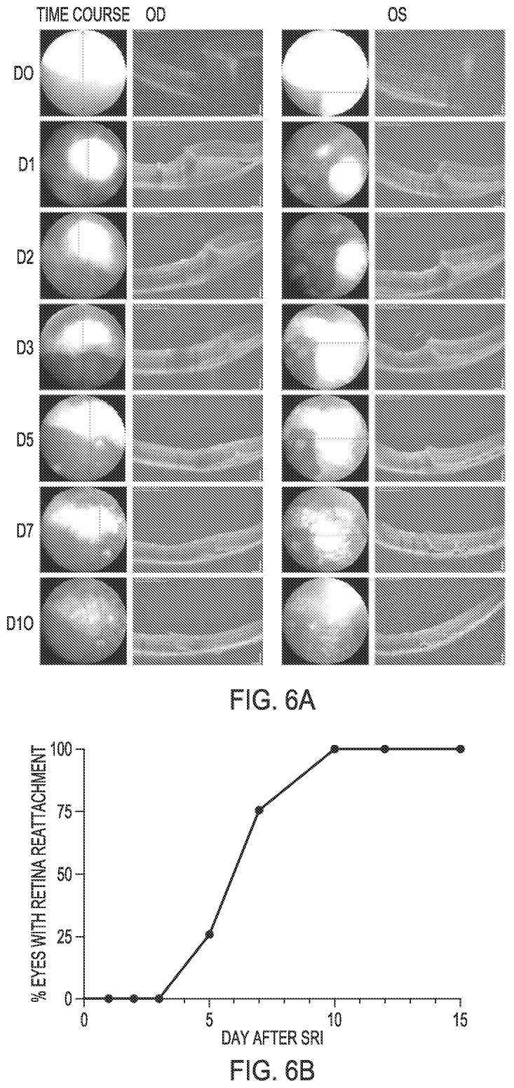

To simulate the clinical course of RRD, wherein most retinas are reattached within a week, a novel model of RRD was developed with spontaneous retinal reattachment (RRD/SRR) by testing a range of lower CMC concentrations (0.01-1%) and volumes (1-4 μl) and monitoring the temporal evolution of the RRD (FIGS. 5, 6). It was found that an SRI volume of 3 μl and concentration of 0.03% resulted in SRR over a period of 5-10 days (FIG. 6B), resembling the usual clinical time course of surgical restoration of retinal apposition to the underlying retinal pigment epithelium and normoxia.

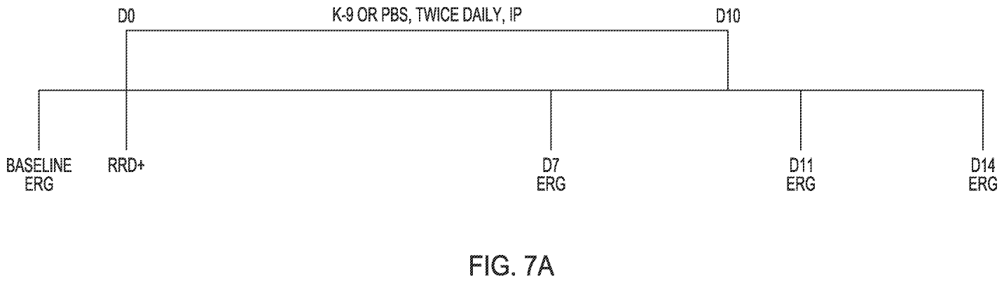

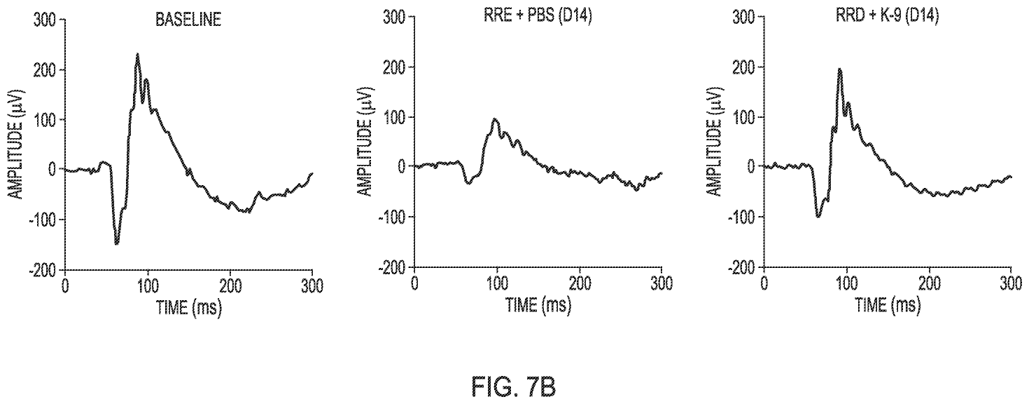

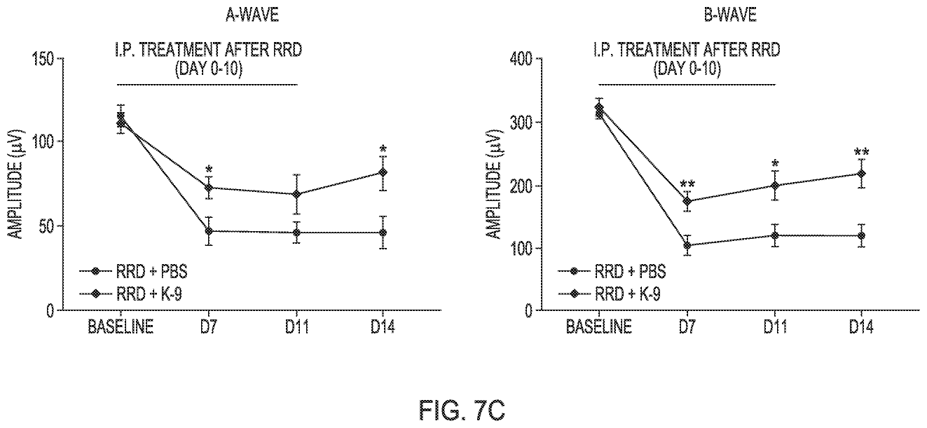

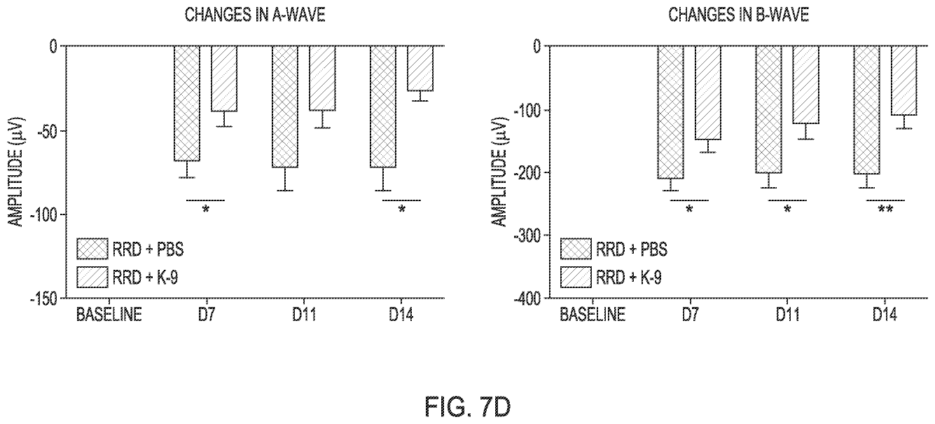

K-9 was then tested in this RRD/SRR model via systemic administration during the first 10 days following RRD (FIG. 7A). It was found that PBS-treated mice displayed larger reductions in the amplitudes of both a- and b-waves over days 7-14, as measured by full-field scotopic ERG, when compared to mice treated with K-9 (FIGS. 7B, 7C). Interestingly, despite anatomic reattachment of the retina, the ERG in PBS-treated animals revealed persistently poor a- and b-wave responses up to 14 days following RRD (FIG. 7C). In contrast, K-9 preserves retinal electrical function during the period of RRD and reattachment.

### Discussion

In RRD, hypoxic and inflammatory signaling induce progressive damage to the retinal ONL (2-6). Consequently, even with successful surgical reattachment, RRD often results in photoreceptor death and lasting functional deficits. (7-9) An interim therapy to protect photoreceptors prior to retinal reattachment may improve visual outcomes. In the present study, K-9 and NRTIs demonstrate anti-inflammatory and neuroprotective effects that may be beneficial in the setting of clinical RRD.

The introduction of a mouse model of RRD created by subretinal injection of a viscous substance (27) has enabled the study of genetic and pharmacological interventions in a tractable platform. These studies reveal that experimental RRD induces inflammasome activation in mice, and that three inflammasome inhibitors (K-9, AZT, and 3TC) reduce TUNEL+ photoreceptor death, a feature that occurs early in the course of retinal detachment in both humans (2) and mice. (27)

Since existing mouse models of RRD result in retinal detachment that persists for several weeks or longer (27), a novel RRD/SRR model was developed in mice. By injecting a lower than typical concentration of CMC, which resembles the gelatinous texture and concentration of human vitreous humor (28), a model was achieved in which spontaneous retinal reattachment occurs over 5 to 10 days of RRD, which approximates the time course of RRD in the standard-of-care clinical setting. (29,30) By self-reattaching, the model avoids reattachment procedures that would be technically complex and likely trauma-inducing in the mouse eye. More importantly, reattachment of the retina facilitates electrophysiologic evaluation and clinically relevant assessments of functional outcomes in RRD.

In this RRD/SRR model, we found that K-9, when administered during the period of retinal detachment, reduced the loss of retinal electrical function during detachment and led to an improvement in ERG parameters following retinal reattachment. These data provide good retinal structure-function correlation and suggest that K-9 has the potential to impart improved visual outcomes in the setting of RRD.

Multiple real-world factors contribute to delayed surgical retinal reattachment, including patient unawareness, difficulty of retinal subspecialty consultation, operating room availability, pandemic-related delays, sociodemographic factors, constraints on physicians' schedules, and the need to stabilize patient health pre-operatively (e.g., blood pressure, glucose levels, and coagulation status). (31-34) In these contexts, and more, K-9, which retains the inflammasome inhibitory activity of the parent NRTI class but lacks the off-target mitochondrial toxicity that plagues NRTIs, (11,20) can provide patients with interim, vision-saving neuroprotection.

### Conclusions

K-9 and NRTIs exhibit anti-inflammatory and neuroprotective activities in experimental RRD. Given its capacity to protect photoreceptor function during the period of RRD and enhance retinal function following reattachment, K-9 is a retinal neuroprotectant Further, this novel RRD/SRR model can facilitate experimental evaluation of functional outcomes relevant to RRD.

### Example II—Kamuvudine-9 Protects Retinal Function

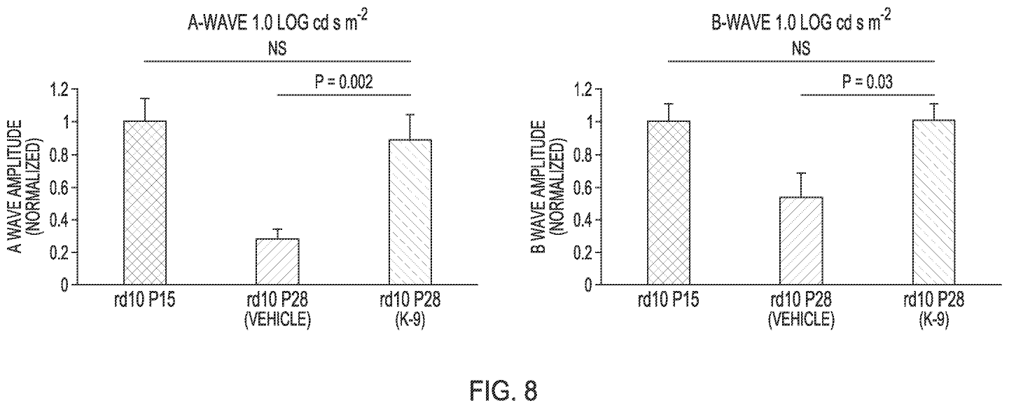

K-9 protects retinal function in the rd10 mouse model of retinal neuronal degeneration. rd10 is an autosomal missense R560C mutation in the Pde6b gene, which results in a recessively inherited retinal neuronal degeneration (FIG. 8). Starting at postnatal day 15 (P15) until P28, rd10 mice were treated with K-9 at 60 mg/kg twice daily via intraperitoneal injection or with an equal volume of vehicle (PBS). Retinal function was monitored with scotopic electroretinography (ERG). Vehicle-treated rd10 mice exhibited a dramatic decline in both the ERG a-wave and b-wave. In contrast, there was completed protection of both the a-wave and b-wave in K9-treated rd10 mice.

### BIBLIOGRAPHY

1. Mitry D, Charteris D G, Fleck B W, Campbell H, Singh J. The epidemiology of rhegmatogenous retinal detachment: geographical variation and clinical associations. Br J Ophthalmol. 2010; 94(6):678-684.
2. Arroyo J G, Yang L, Bula D, Chen D F. Photoreceptor apoptosis in human retinal detachment. Am J Ophthalmol. 2005; 139(4):605-610.
3. Cook B, Lewis G P, Fisher S K, Adler R. Apoptotic photoreceptor degeneration in experimental retinal detachment. Invest Ophthalmol Vis Sci. 1995; 36(6):990-996.
4. Mervin K, Valter K, Maslim J, Lewis G, Fisher S, Stone J. Limiting photoreceptor death and deconstruction during experimental retinal detachment: the value of oxygen supplementation. Am J Ophthalmol. 1999; 128(2):155-164.
5. Hisatomi T, Sakamoto T, Goto Y, et al. Critical role of photoreceptor apoptosis in functional damage after retinal detachment. Curr Eye Res. 2002; 24(3):161-172.
6. Kataoka K, Matsumoto H, Kaneko H, et al. Macrophage- and RIP3-dependent inflammasome activation exacerbates retinal detachment-induced photoreceptor cell death. Cell Death Dis. 2015; 6(4):e1731.
7. Ross W H. Visual recovery after macula-off retinal detachment. Eye (Lond). 2002; 16(4):440-446.
8. Burton T C. Recovery of visual acuity after retinal detachment involving the macula. Trans Am Ophthalmol Soc. 1982; 80:475-497.
9. Williamson T H, Shunmugam M, Rodrigues I, Dogramaci M, Lee E. Characteristics of rhegmatogenous retinal detachment and their relationship to visual outcome. Eye (Lond). 2013; 27(9):1063-1069.
10. Broz P, Dixit V M. Inflammasomes: mechanism of assembly, regulation and signalling. Nat Rev Immunol. 2016; 16(7):407-420.
11. Fowler B J, Gelfand B D, Kim Y, et al. Nucleoside reverse transcriptase inhibitors possess intrinsic anti-inflammatory activity. Science. 2014; 346(6212):1000-1003.
12. Ambati J, Magagnoli J, Leung H, et al. Repurposing anti-inflammasome NRTIs for improving insulin sensitivity and reducing type 2 diabetes development. Nat Commun. 2020; 11(1):4737.
13. Pavlou S, Augustine J, Cunning R, et al. Attenuating Diabetic Vascular and Neuronal Defects by Targeting P2rx7. Int J Mol Sci. 2019; 20(9):2101.
14. Kong H, Zhao H, Chen T, Song Y, Cui Y. Targeted P2X7/NLRP3 signaling pathway against inflammation, apoptosis, and pyroptosis of retinal endothelial cells in diabetic retinopathy. Cell Death Dis. 2022; 13(4):336.
15. De Cecco M, Ito T, Petrashen A P, et al. L1 drives IFN in senescent cells and promotes age-associated inflammation. Nature. 2019; 566(7742):73-78.
16. Mizutani T, Fowler B J, Kim Y, et al. Nucleoside Reverse Transcriptase Inhibitors Suppress Laser-Induced Choroidal Neovascularization in Mice. Invest Ophthalmol Vis Sci. 2015; 56(12):7122-7129.
17. Narendran S, Pereira F, Yerramothu P, et al. A Clinical Metabolite of Azidothymidine Inhibits Experimental Choroidal Neovascularization and Retinal Pigmented Epithelium Degeneration. Invest Ophthalmol Vis Sci. 2020; 61(10):4.
18. Fukuda S, Narendran S, Varshney A, et al. Alu complementary DNA is enriched in atrophic macular degeneration and triggers retinal pigmented epithelium toxicity via cytosolic innate immunity. Sci Adv. 2021; 7(40):eabj3658.
19. Yamada K, Kaneko H, Shimizu H, et al. Lamivudine Inhibits Alu RNA-induced Retinal Pigment Epithelium Degeneration via Anti-inflammatory and Anti-senescence Activities. Transl Vis Sci Technol. 2020; 9(8):1.
20. Koczor C A and Lewis W. Nucleoside reverse transcriptase inhibitor toxicity and mitochondrial DNA. Expert Opin Drug Metab Toxicol 2010; 6:1493-1504.
21. Narendran S, Pereira F, Yerramothu P, et al. Nucleoside reverse transcriptase inhibitors and Kamuvudines inhibit amyloid-β induced retinal pigmented epithelium degeneration. Signal Transduct Target Ther. 2021; 6(1):149.
22. Azarmina M, Moradian S, Azarmina H. Electroretinographic changes following retinal reattachment surgery. J Ophthalmic Vis Res. 2013; 8(4):321-329.
23. Huang P, Narendran S, Pereira F, et al. Subretinal injection in mice to study retinal physiology and disease. Nat Protoc. 2022; 17(6):1468-1485.
24. Maidana D E, Tsoka P, Tian B, et al. A Novel ImageJ Macro for Automated Cell Death Quantitation in the Retina. Invest Ophthalmol Vis Sci. 2015; 56(11):6701-6708.
25. Watanabe S, Usui-Kawanishi F, Karasawa T, et al. Glucose regulates hypoxia-induced NLRP3 inflammasome activation in macrophages. J Cell Physiol. 2020; 235(10):7554-7566.
26. Jiang Q, Geng X, Warren J, et al. Hypoxia Inducible Factor-1α (HIF-1α) Mediates NLRP3 Inflammasome-Dependent-Pyroptotic and Apoptotic Cell Death Following Ischemic Stroke. Neuroscience. 2020; 448:126-139.
27. Yang L, Bula D, Arroyo J G, Chen D F. Preventing retinal detachment-associated photoreceptor cell loss in Bax-deficient mice. Invest Ophthalmol Vis Sci. 2004; 45(2):648-654.
28. Bishop P. The biochemical structure of mammalian vitreous. Eye (Lond). 1996; 10 (Pt 6):664-670.
29. Elghawy O, Duong R, Nigussie A, Bogaard J D, Patrie J, Shildkrot Y. Effect of surgical timing in 23-g pars plana vitrectomy for primary repair of macula-off rhegmatogenous retinal detachment, a retrospective study. BMC Ophthalmol. 2022; 22(1):136.
30. Lee C S, Shaver K, Yun S H, Kim D, Wen S, Ghorayeb G. Comparison of the visual outcome between macula-on and macula-off rhegmatogenous retinal detachment based on the duration of macular detachment. BMJ Open Ophthalmol. 2021; 6(1):e000615.
31. Goezinne F, La Heij E C, Berendschot T T, et al. Patient ignorance is the main reason for treatment delay in primary rhegmatogenous retinal detachment in The Netherlands. Eye (Lond). 2009; 23(6):1393-1399.
32. Eijk E S, Busschbach J J, Timman R, Monteban H C, Vissers J M, van Meurs J C. What made you wait so long? Delays in presentation of retinal detachment: knowledge is related to an attached macula. Acta Ophthalmol. 2016; 94(5):434-440.
33. Tolou C, Mahieu-Durringer L, Cassagne M, et al. Délai de prise en charge des patients atteints d'un premier épisode de décollement de rétine sur l'œil étudié en Midi-Pyrénées [Treatment delay in patients with first episode of retinal detachment in the studied eye in Midi-Pyrénées]. J Fr Ophtalmol. 2016; 39(1):90-97.
34. Xu D, Uhr J, Patel S N, et al. Sociodemographic Factors Influencing Rhegmatogenous Retinal Detachment Presentation and Outcome. Ophthalmol Retina. 2021; 5(4):337-341.

One of ordinary skill in the art will recognize that additional embodiments or implementations are possible without departing from the teachings of the present disclosure or the scope of the claims which follow. This detailed description, and particularly the specific details of the exemplary embodiments and implementations disclosed herein, is given primarily for clarity of understanding, and no unnecessary limitations are to be understood therefrom, for modifications will become obvious to those skilled in the art upon reading this disclosure and may be made without departing from the scope of the claimed invention.

All publications, patents, and patent applications, Genbank sequences, websites and other published materials referred to throughout the disclosure herein are herein incorporated by reference to the same extent as if each individual publication, patent, or patent application, Genbank sequences, websites and other published materials was specifically and individually indicated to be incorporated by reference. In the event that the definition of a term incorporated by reference conflicts with a term defined herein, this specification shall control.

1. A method to treat rhegmatogenous retinal detachment (RRD) and/or improve visual outcomes of RRD comprising administering to a subject in need thereof one or more inflammasome inhibitors.

2. A method for neuroprotection of photoreceptors comprising administering to a subject with retinal detachment one or more inflammasome inhibitors.

3. (canceled)

4. A method to preserve and/or improve retinal function comprising administering to a subject with retinal detachment one or more inflammasome inhibitors.

5. The method of claim 4, wherein retinal function is measured by electroretinography (ERG).

6. The method of claim 1, wherein the subject has complete or partial retinal detachment.

7. The method of claim 5, wherein the subject undergoes surgery for retinal detachment after administration of the one or more inflammasome inhibitors.

8. The method of claim 1, wherein the one or more inflammasome inhibitors are administered orally or injected.

9. The method of claim 8, wherein the one or more inflammasome inhibitors are injected into an eye of the subject.

10. The method of claim 9, wherein the one or more inflammasome inhibitors are injected into vitreous humor of the eye of the subject.

11. The method of claim 1, wherein one or more inflammasome inhibitors comprises a compound of structural Formula (I) 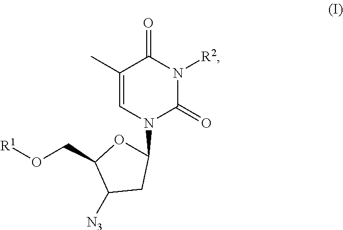
wherein:
R¹ is C₁₋₄ alkyl; and
R² is H or C₁₋₄ alkyl, provided that when R² is H, R¹ is not CH₃
or a salt thereof.

12. The method of claim 11, wherein R² is CH₃ or CH₂CH₃.

13. The method of claim 11, wherein R¹ is n-C₄H₉.

14. The method of claim 1, wherein the one or more inflammasome inhibitors comprises one or more of 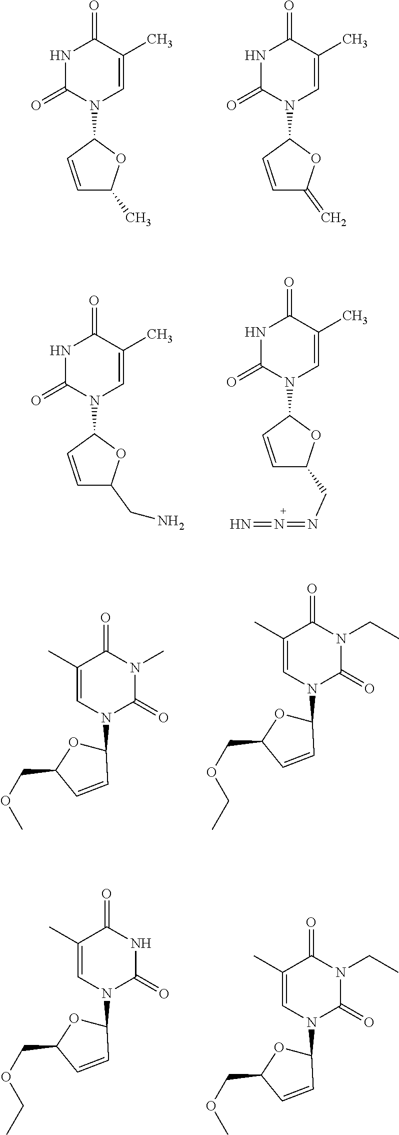 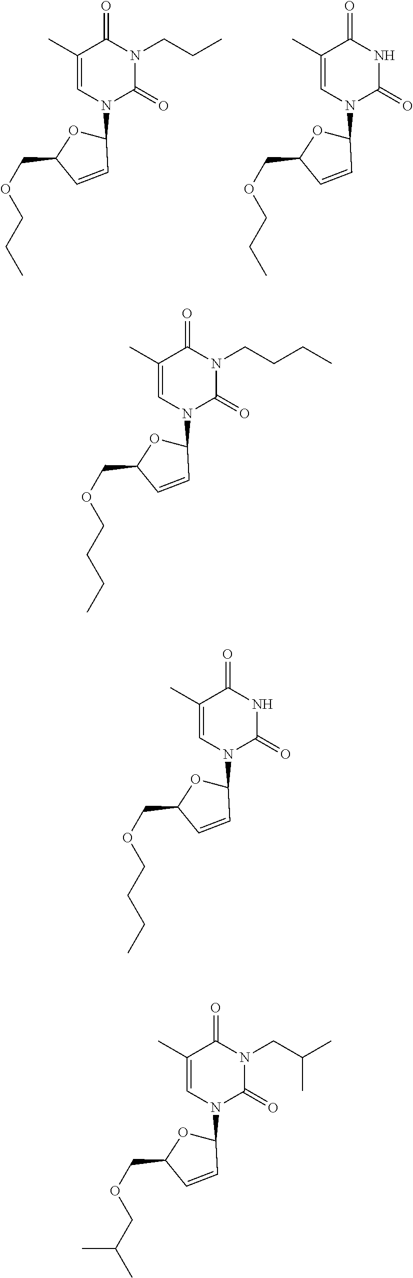 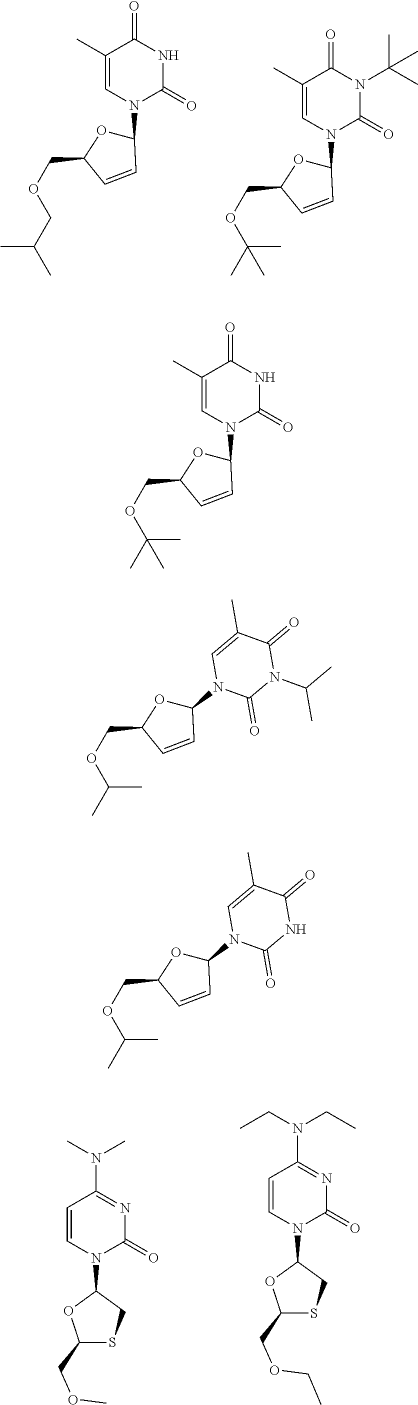 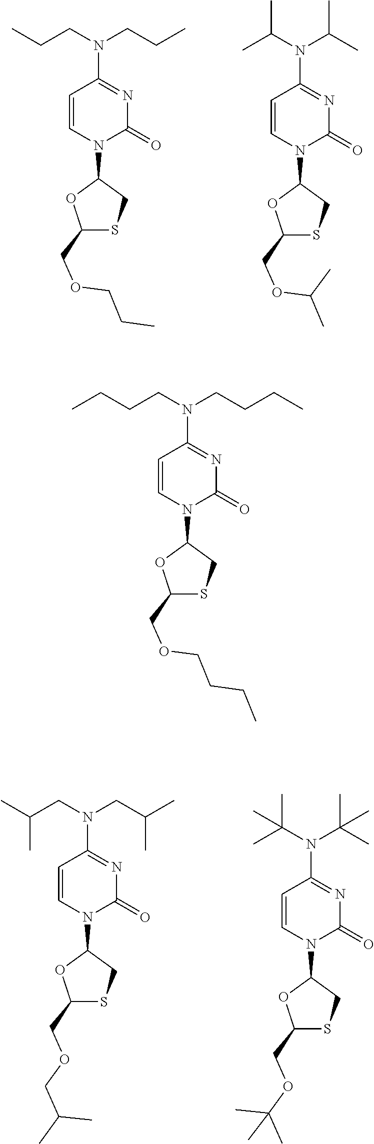 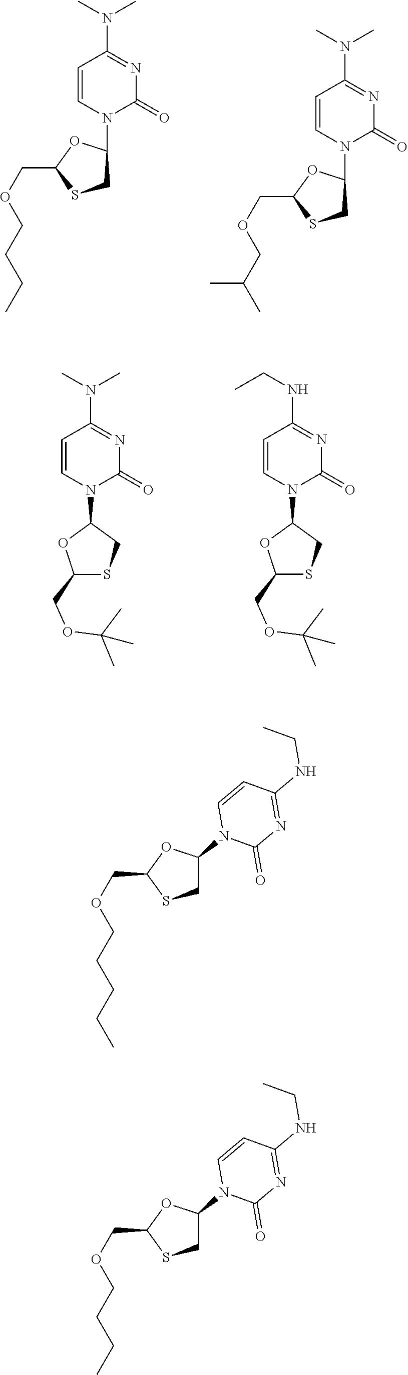 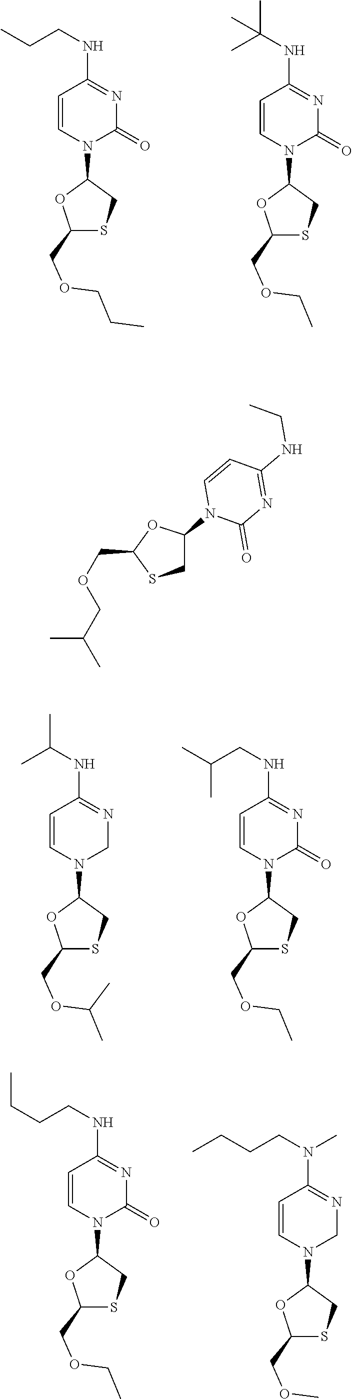 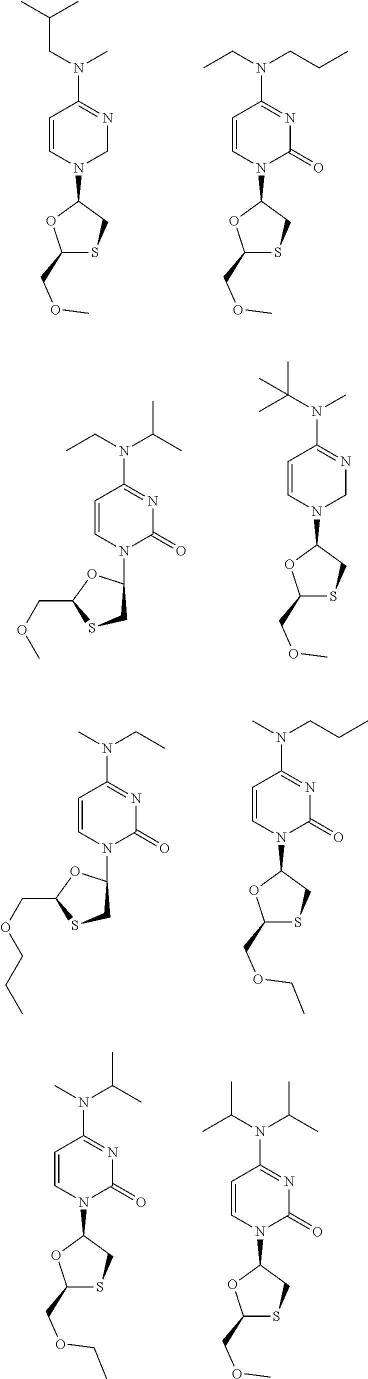 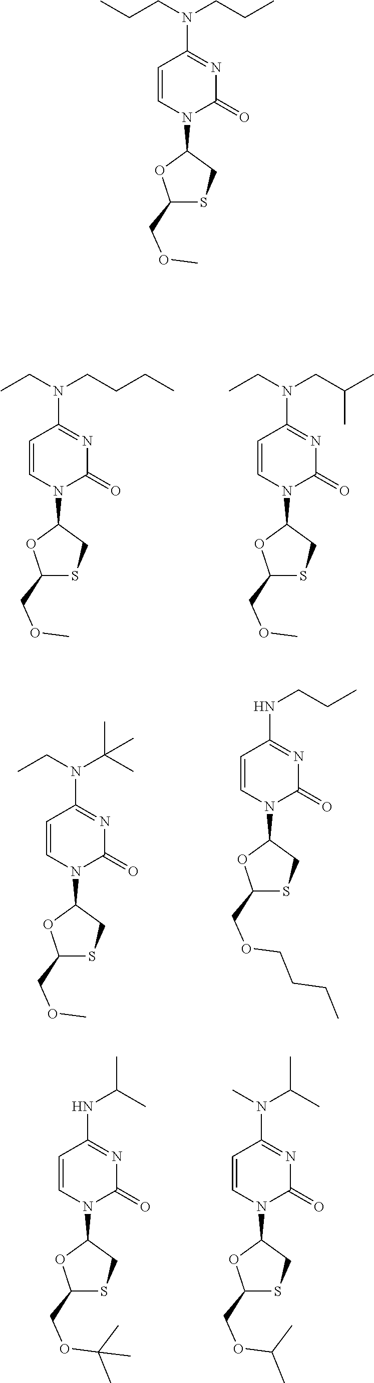 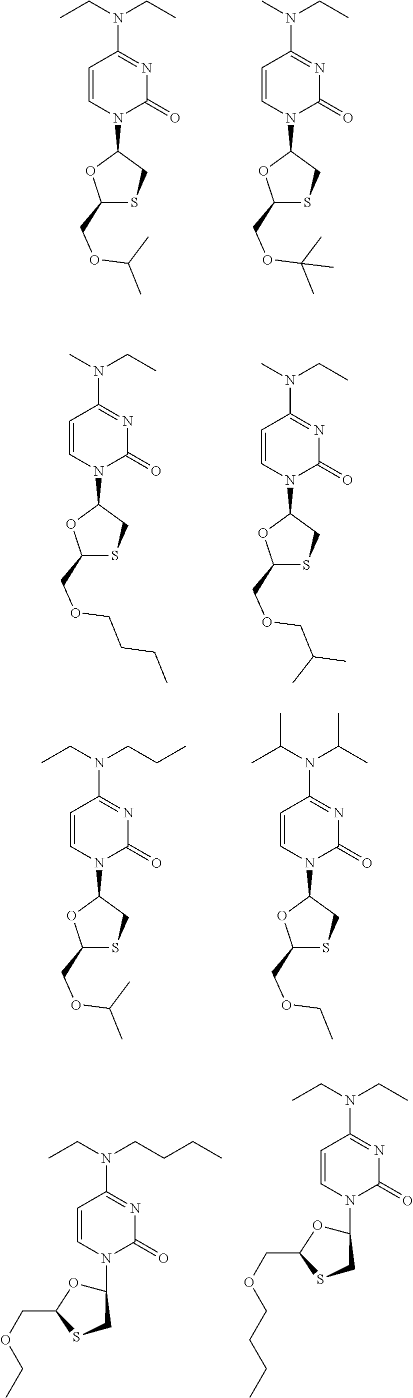 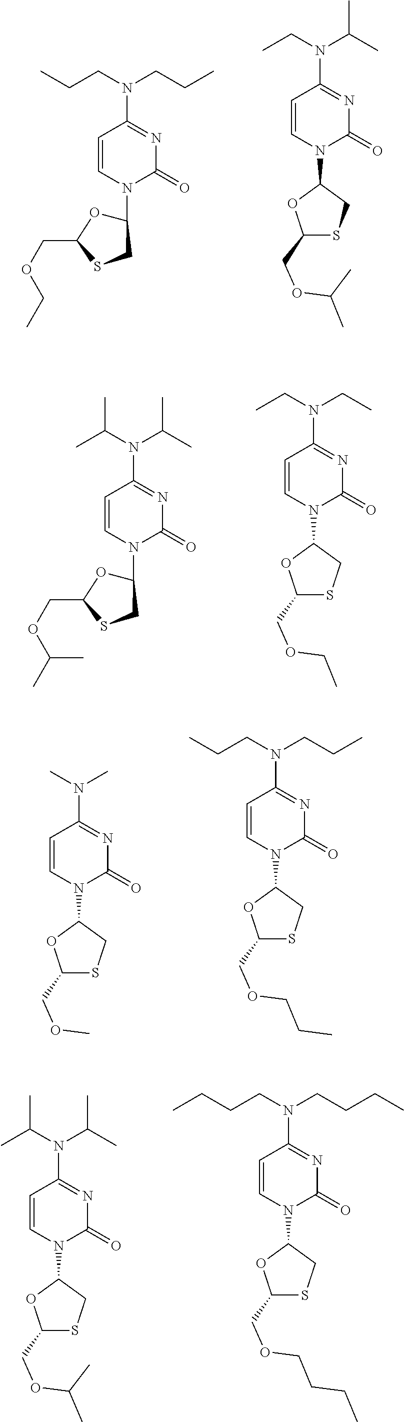 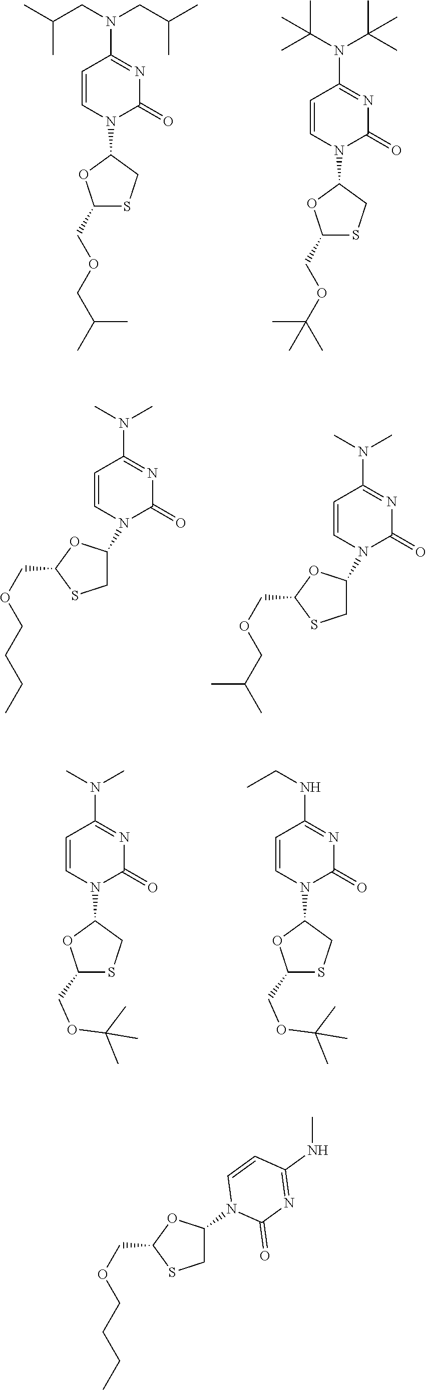 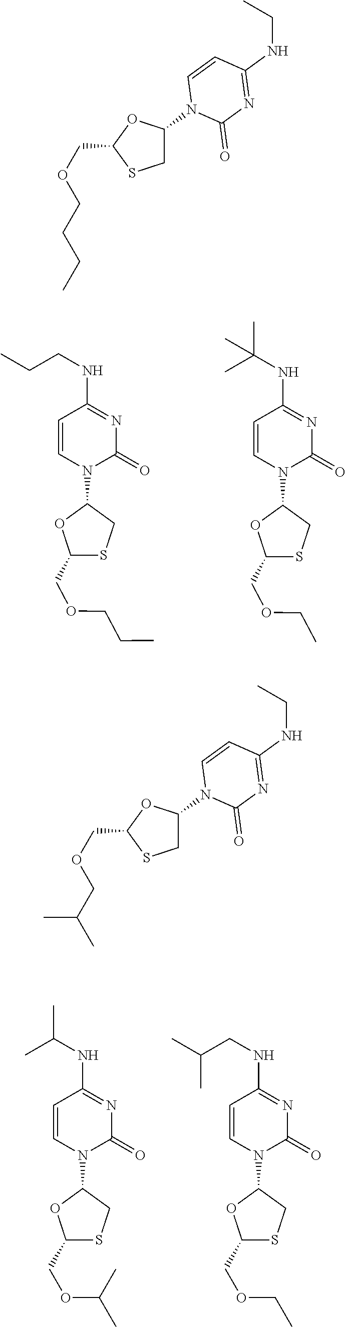 [SMILES: CCCCOC[C@@H]1O[C@H](n2ccc(NCC)nc2=O)CS1 ; CCCNc1ccn([C@@H]2CS[C@H](COCCC)O2)c(=O)n1 ; CCOC[C@@H]1O[C@H](n2ccc(NC(C)(C)C)nc2=O)CS1 ; CCNc1ccn([C@@H]2CS[C@H](COCC(C)C)O2)c(=O)n1 ; CC(C)Nc1ccn([C@@H]2CS[C@H](COC(C)C)O2)c(=O)n1 ; CCOC[C@@H]1O[C@H](n2ccc(NCC(C)C)nc2=O)CS1] 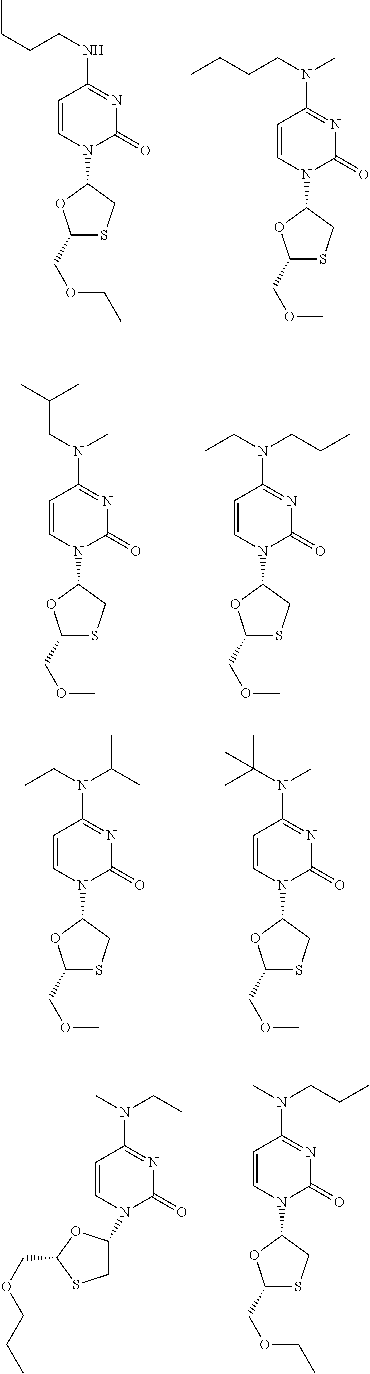 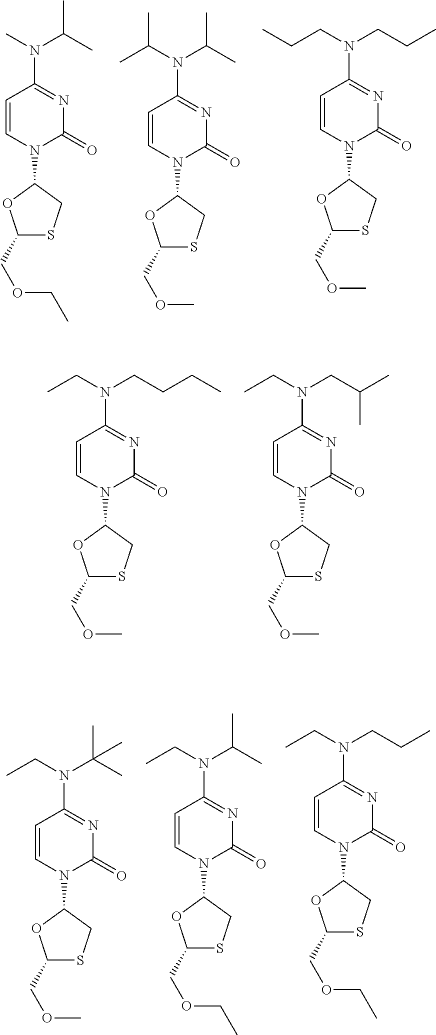 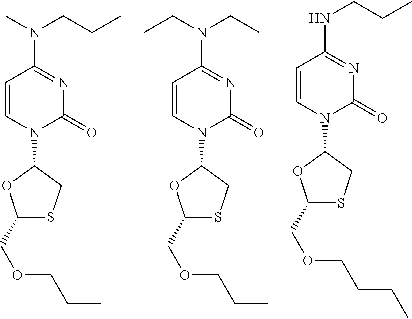 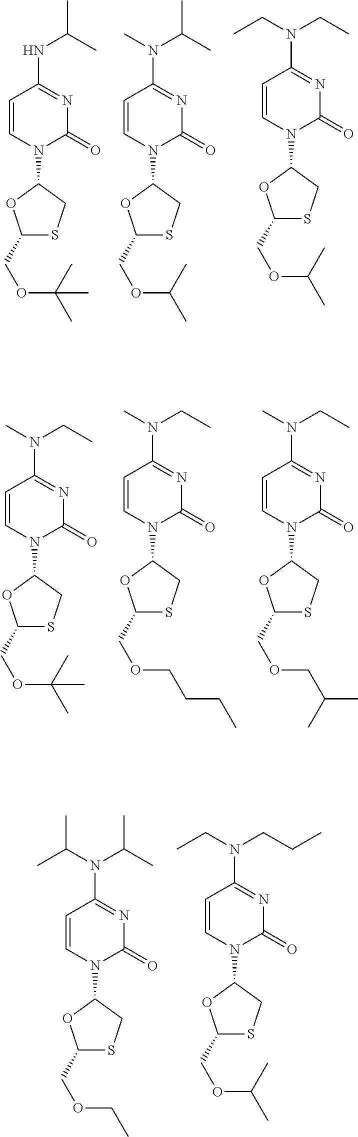 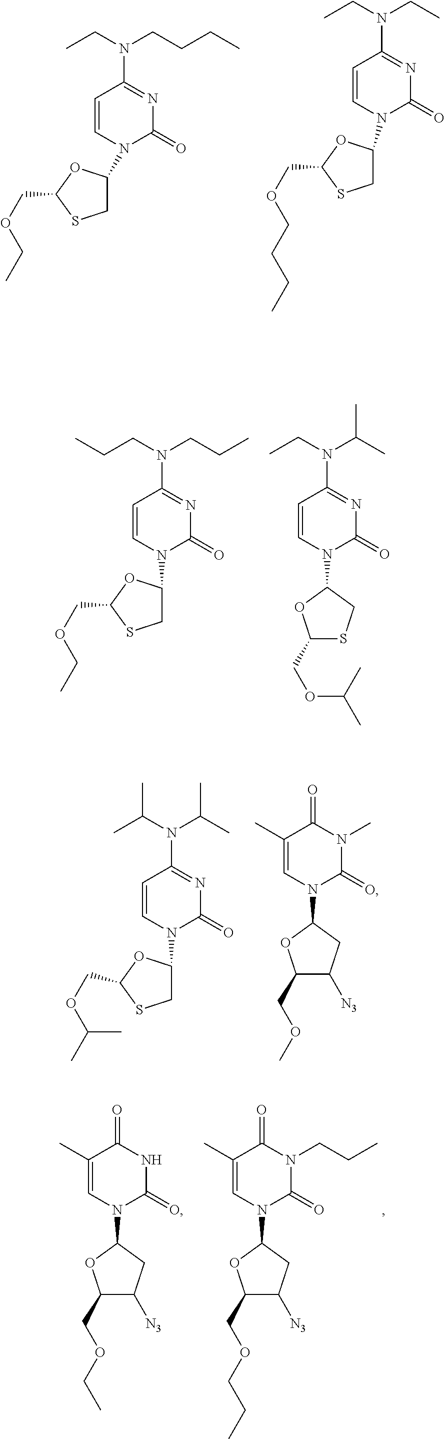 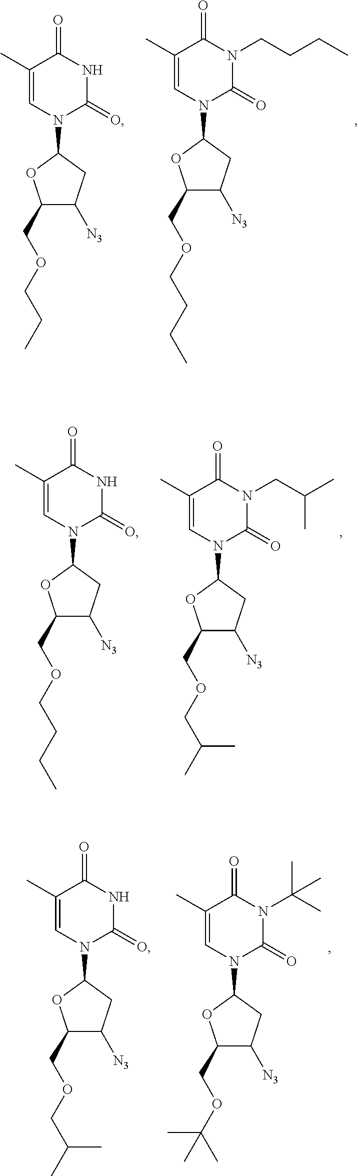 [SMILES: CCCOC[C@H]1O[C@@H](n2cc(C)c(=O)[nH]c2=O)CC1N=[N+]=[N-] ; CCCCOC[C@H]1O[C@@H](n2cc(C)c(=O)n(CCCC)c2=O)CC1N=[N+]=[N-] ; CCCCOC[C@H]1O[C@@H](n2cc(C)c(=O)[nH]c2=O)CC1N=[N+]=[N-] ; Cc1cn([C@H]2CC(N=[N+]=[N-])[C@@H](COCC(C)C)O2)c(=O)n(CC(C)C)c1=O ; Cc1cn([C@H]2CC(N=[N+]=[N-])[C@@H](COCC(C)C)O2)c(=O)[nH]c1=O ; Cc1cn([C@H]2CC(N=[N+]=[N-])[C@@H](COC(C)(C)C)O2)c(=O)n(C(C)(C)C)c1=O] 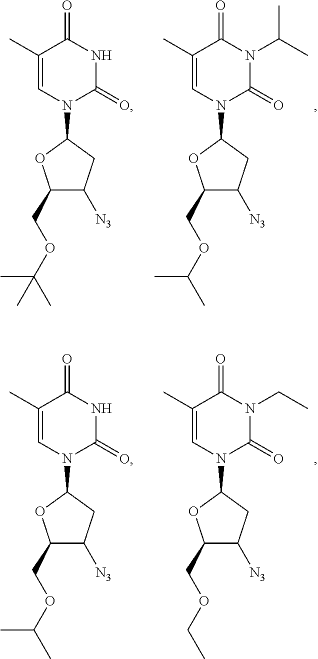 [SMILES: Cc1cn([C@H]2CC(N=[N+]=[N-])[C@@H](COC(C)(C)C)O2)c(=O)[nH]c1=O ; Cc1cn([C@H]2CC(N=[N+]=[N-])[C@@H](COC(C)C)O2)c(=O)n(C(C)C)c1=O ; Cc1cn([C@H]2CC(N=[N+]=[N-])[C@@H](COC(C)C)O2)c(=O)[nH]c1=O ; CCOC[C@H]1O[C@@H](n2cc(C)c(=O)n(CC)c2=O)CC1N=[N+]=[N-]]
an enantiomer or a pharmaceutically acceptable salt thereof.

15. The method of claim 1, wherein the one or more inflammasome inhibitors are Kamuvudine-9 (K-9), lamivudine (3TC), azidothymidine (AZT), a salt thereof or a combination thereof.

16. The method of claim 1, wherein the one or more inflammasome inhibitors are 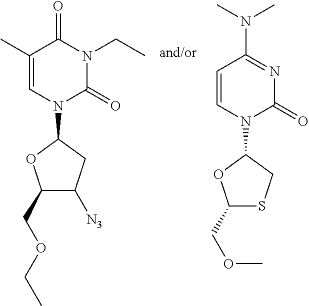 [SMILES: CCOC[C@H]1O[C@@H](n2cc(C)c(=O)n(CC)c2=O)CC1N=[N+]=[N-] ; COC[C@@H]1O[C@H](n2ccc(N(C)C)nc2=O)CS1]
an enantiomer or pharmaceutically acceptable salt thereof.

17.-18. (canceled)
